(19) United States
(12) Patent Application Publication
(10) Pub. No.: US 2005/0033116 A1
(43) Pub. Date: Feb. 10, 2005

(54) Endoscope apparatus

(76) Inventors: Kiyoshi Miyake, Asaka-shi (JP); Mitsuo Obata, Tokyo (JP)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/842,190

(22) Filed: May 10, 2004

(30) Foreign Application Priority Data
May 12, 2003 (JP) 2003-133345

(51) Int. Cl. A61B 1/04

(52) U.S. Cl. 600/109; 600/118

(57) ABSTRACT

An endoscope apparatus comprises a scope main body which has an insertion section inserted into a specimen, a box body section which is used in combination with the scope main body, and an amplification device which is provided between the scope main body and the box body section and amplifies the normal function of at least one of the scope main body and the box body section.

### BACKGROUND OF THE INVENTION

1. Field of the Invention

The present invention relates to an endoscope apparatus having an endoscope which is detachably connected to an endoscope detach section of the apparatus main body.

Priority is claimed on Japanese Patent Application No. 2003-133345, filed May 12, 2003, the content of which is incorporated herein by reference.

2. Description of Related Art

Generally, in an endoscope apparatus there is provided a fixed unit of the apparatus main body which has a built-in light source such as an illumination light, and an endoscope which is inserted into the space of the subject of examination and is actually used for the inside examination. A connector receiver for connecting the endoscope is provided on the fixed unit. On the endoscope, an operation unit on the handle side is placed at the base of a long and thin insertion section which is inserted into the space of the subject of examination. One end of a universal cord is connected to the operation unit. The other end of the universal cord is connected to a connector unit. A connector receiver is provided on the fixed unit of the apparatus main body. Moreover, the endoscope is used in a state where the connector unit is detachably connected to the connector receiver of the fixed unit.

A video scope which is combined with an imager such as a CCD in the observational optical system of the endoscope, is shown in Japanese Unexamined Patent Application, First Publication No. H05-211988. A light guide connector for optical connections and an electrical connector for electrical connections are provided on the end of a universal cord which is connected to the operation unit on the handle side in the video scope. Furthermore, a light source apparatus, a camera control unit (CCU) and the like are provided on the apparatus main body side of the fixed unit. A connector receiver for optical connections is provided on the light source apparatus, and a connector receiver for electrical connections is provided on the CCU. Moreover, the endoscope is used under a state set where a light guide connector of the endoscope is connected to the connector receiver for optical connections of the light source apparatus, and the electrical connector for electrical connections is connected to the connector receiver for electrical connections of the CCU.

An endoscope system having an integrated structure where an insertion section of the endoscope, a light source apparatus, and a processor are integrally assembled and built-in, is shown in Japanese Unexamined Patent Application, First Publication No.2001-330783.

An endoscope system having a structure where an imager driving circuit and a signal processing circuit constituting part of a CCU, are separated as the imager corresponding units, and that part is made detachable with the CCU main body, is shown in Japanese Unexamined Patent Application, First Publication No. H06-70883.

An endoscope system having a structure where a module is detachably connected to the tip end of an insertion section of an endoscope, then a second module (sensitizing module) is detachably connected between the module and the tip end of the endoscope, and the second module comprises an amplification circuit, is shown in Japanese Patent Publication No. 3270106.

### SUMMARY OF THE INVENTION

An endoscope apparatus of the present invention includes: a scope main body which has an insertion section for insertion into a specimen; a box body section which is used in combination with the scope main body; and an amplification device which is provided between the scope main body and the box body section, and amplifies the normal function of at least one of the scope main body and the box body section.

### BRIEF DESCRIPTION OF THE DRAWINGS

FIG. 1 is an overall schematic block diagram showing a state where a fixed unit is combined with a scope unit without an amplification unit, in an industrial endoscope apparatus of a first embodiment of the present invention.

FIG. 2 is an overall schematic block diagram showing a state where the fixed unit is combined with the scope unit through the amplification unit, in the industrial endoscope apparatus of the first embodiment.

FIG. 3 is a perspective view showing a case main body of the industrial endoscope apparatus of the first embodiment.

FIG. 4 is an exploded perspective view showing a fixed unit and two types of scope unit before assembling, in the industrial endoscope apparatus of the first embodiment.

FIG. 5 is a perspective view showing a state where optical adapters are detached from the tip end of an insertion section of a scope main body, in the industrial endoscope apparatus of the first embodiment.

FIG. 6 is a vertical sectional view showing the inner structure of the tip end of the insertion section of the scope main body, in the industrial endoscope apparatus of the first embodiment.

FIG. 7 is a schematic block diagram of a CCU control unit in the industrial endoscope apparatus of the first embodiment.

FIG. 8 is a perspective view showing a state where the fixed unit is combined with the scope unit without the amplification unit and used, in the industrial endoscope apparatus of the first embodiment.

FIG. 9 is a perspective view showing a state where the fixed unit is combined with the scope unit through the amplification unit and used, in the industrial endoscope apparatus of the first embodiment.

FIG. 10A to FIG. 10C show a base unit of the scope unit. FIG. 10A is a side view. FIG 10B is a front view. FIG. 10C is a sectional view along the line XC-XC of FIG. 10B.

FIG. 11A is a vertical sectional view of the main parts showing the attaching state of an optical connector on the light source side of the industrial endoscope apparatus of the first embodiment.

FIG. 11B is a vertical sectional view of the main parts showing the attaching state of an LG connector of the scope unit of the industrial endoscope apparatus of the first embodiment.

FIG. 12A is a plan view showing a connector receiver.

FIG. 12B is a perspective view showing the LG connector.

FIG. 13A to FIG. 13C show a state where an electrical connector main body on the scope side is mounted on a substrate of the industrial endoscope apparatus of the first embodiment. FIG. 13A is a plan view. FIG. 13B is a front view. FIG. 13C is a sectional view along the line 13C-13C of FIG. 13B.

FIG. 14A to FIG. 14C show a state where an electrical connector main body on the fixed unit side is mounted on the substrate of the industrial endoscope apparatus of the first embodiment. FIG. 14A is a plan view. FIG. 14B is a front view. FIG. 14C is a sectional view along the line 14C-14C of FIG. 14B.

FIG. 15 is a vertical sectional view showing a state where the fixed unit is combined with the scope unit without the amplification unit and used, in the industrial endoscope apparatus of the first embodiment.

FIG. 16A to FIG. 16C show a fixing device for fixing of the fixed unit and the scope unit to the base unit, of the industrial endoscope apparatus of the first embodiment. FIG. 16A is a vertical sectional view of the main parts showing the attaching state of the fixing device. FIG. 16B is a plan view showing a fixed spring member of the fixed unit. FIG. 16C is a perspective view showing a locking groove of a shaft of the fixing device.

FIG. 17 is a vertical sectional view showing a state where the fixed unit is combined with the scope unit through the amplification unit and used, in the industrial endoscope apparatus of the first embodiment.

FIG. 18A to FIG. 18C are diagrams of when the fixed unit is combined with the scope unit through the amplification unit and used, in the industrial endoscope apparatus of the first embodiment. FIG. 18A is a vertical sectional view of the main parts showing the attaching state of the fixing device. FIG. 18B is a plan view showing a fixed spring member of the fixed unit. FIG. 18C is a perspective view showing a locking groove of the shaft of the fixing device.

FIG. 19A is a vertical sectional view of the main parts showing the attaching state of an amplification side female type optical connector unit of the amplification unit, of the industrial endoscope apparatus of the first embodiment.

FIG. 19B is a vertical sectional view of the main parts showing the attaching state of the LG connector of the scope unit, of the industrial endoscope apparatus of the first embodiment.

FIG. 20A is a vertical sectional view of the main parts showing the attaching state of an optical connector on the light source side of the fixed unit, of the industrial endoscope apparatus of the first embodiment.

FIG. 20B is a vertical sectional view of the main parts showing the attaching state of an amplification side male type optical connector unit of the amplification unit, of the industrial endoscope apparatus of the first embodiment.

FIG. 21A to FIG. 21C show a state where an amplification side female type electrical connector main body of the industrial endoscope apparatus of the first embodiment is mounted on a substrate. FIG. 21A is a plan view. FIG. 21B is a front view. FIG. 21C is a sectional view along the line 21C-21C of FIG. 21B.

FIG. 22A to FIG. 22C show a state where an amplification side male type electrical connector main body of the industrial endoscope apparatus of the first embodiment is mounted on the substrate. FIG. 22A is a plan view. FIG. 22B is a front view. FIG. 22C is a sectional view along the line 22C-22C of FIG. 22B.

FIG. 23 is a block diagram showing the inner structure of an amplification circuit section of the industrial endoscope apparatus of the first embodiment.

FIG. 24 is a flowchart for explaining the operation of the amplification unit when starting up the industrial endoscope apparatus of the first embodiment.

FIG. 25 is an overall schematic block diagram showing a state where a fixed unit is combined with a scope unit without an amplification unit, in an industrial endoscope apparatus of a second embodiment of the present invention.

FIG. 26 is a vertical sectional view showing the inner structure of the junction of the fixed unit, the amplification unit and the scope unit, of the industrial endoscope apparatus of the second embodiment.

FIG. 27A is a vertical sectional view of the main parts showing the attaching state of the LG connector of the amplification unit, of the industrial endoscope apparatus of the second embodiment.

FIG. 27B is a sectional view along the line 27B-27B of FIG. 27A.

FIG. 28 is an overall schematic block diagram showing the inner structure of an industrial endoscope apparatus of a third embodiment of the present invention.

FIG. 29 is a perspective view showing the main parts structure of a pair of curve operation mechanisms of a scope unit, of the industrial endoscope apparatus of the third embodiment.

FIG. 30 is a perspective view showing the inner structure of an electrical curve drive unit of an amplification unit, of the industrial endoscope apparatus of the third embodiment.

FIG. 31 is a perspective view showing a modified example of a pair of the curve operation mechanisms of the scope unit, of the industrial endoscope apparatus of the third embodiment.

FIG. 32 is an overall schematic block diagram showing the inner structure of an industrial endoscope apparatus of a fourth embodiment of the present invention.

FIG. 33 is an overall schematic block diagram showing the inner structure of an industrial endoscope apparatus of a fifth embodiment of the present invention.

FIG. 34 is a perspective view showing a state where a storage section box is detached from an assembly unit comprising a fixed unit, a scope unit and an amplification unit, of the industrial endoscope apparatus of the fifth embodiment.

FIG. 35 is a perspective view showing a state where the insertion of the scope unit is stored into an insertion storage section of the amplification unit, of the industrial endoscope apparatus of the fifth embodiment.

FIG. 36A shows a sixth embodiment of the present invention, being a perspective view showing a state where the storage section box is detached from the assembly unit comprising the fixed unit and a first scope unit.

FIG. 36B shows the sixth embodiment of the present invention and is a perspective view showing a state where the storage section box is assembled with the assembly unit comprising the fixed unit and the first scope unit.

FIG. 37A is a perspective view showing a state where the storage section box is detached from the assembly unit comprising the fixed unit, a second scope unit and the amplification unit, of the sixth embodiment.

FIG. 37B is a perspective view showing a state where the storage section box is assembled with the assembly unit comprising the fixed unit, the second scope unit and the amplification unit, of the sixth embodiment.

FIG. 38 is an overall schematic block diagram showing the inner structure of an industrial endoscope apparatus of a seventh embodiment of the present invention.

### DETAILED DESCRIPTION OF THE INVENTION

(First Embodiment)

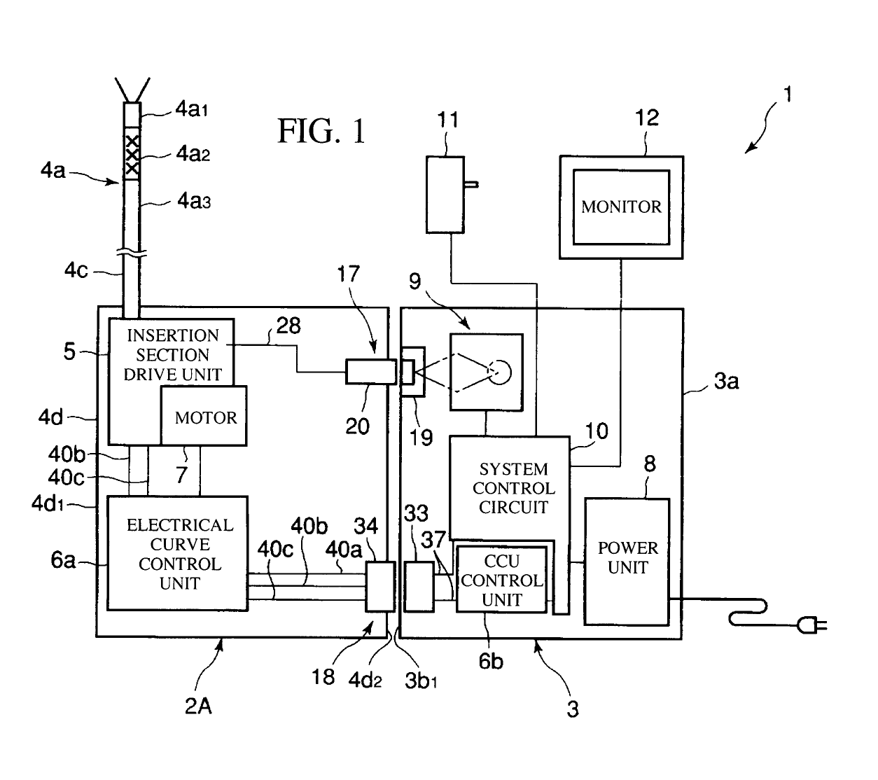

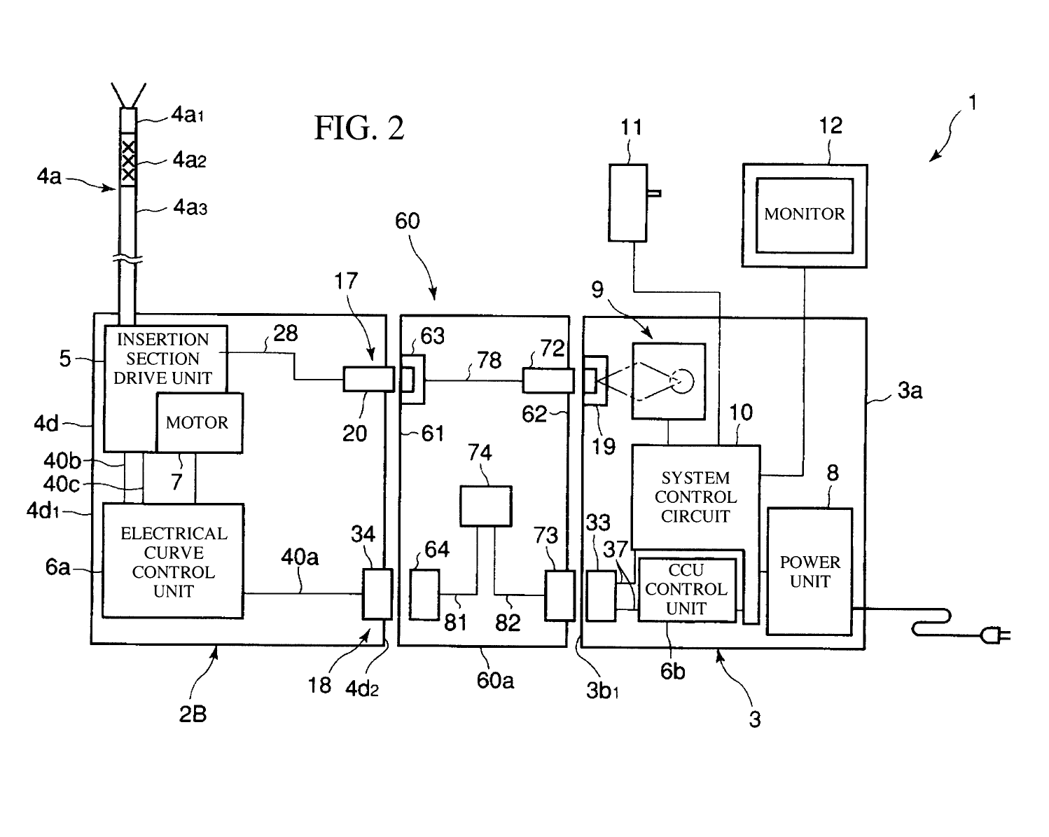

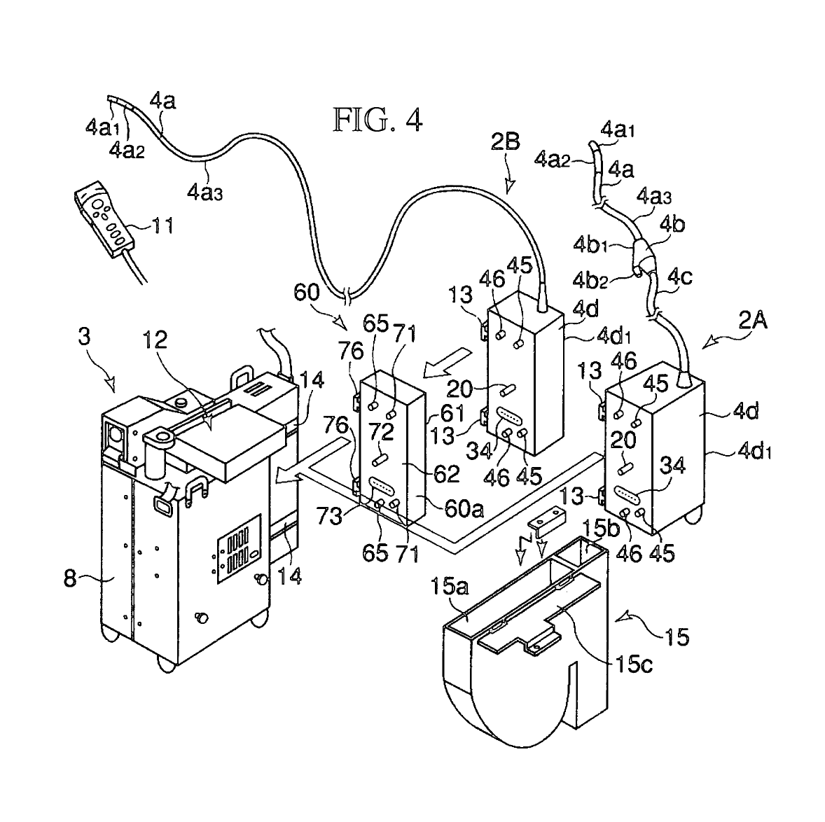

Hereunder a first embodiment of the present invention will be described, referring to FIG. 1 to FIG. 24. FIG. 1 is a schematic block diagram of a system for an industrial endoscope apparatus 1 of the present embodiment. The system of the endoscope apparatus 1 is mainly provided with; a scope unit 2 (a scope main body), a fixed unit 3 (a box body section) and an amplification unit 60 (refer to FIG. 2) which will be described later. Furthermore, the system of the endoscope apparatus 1 of the present embodiment is provided in advance with a plurality of different models, for example in the system in FIG. 4 with two models of the scope unit (the first scope unit 2A and the second scope unit 2B). Also, the fixed unit 3 of the present embodiment is a single unit which can be used in common with a plurality of models of the scope units 2A and 2B.

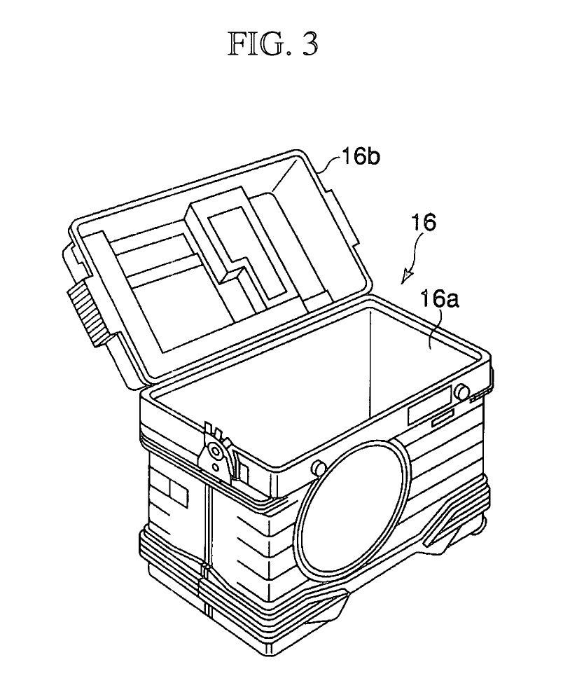

As shown in FIG. 1, when the first scope unit 2A is used the first scope unit 2A is connected directly to the fixed unit 3. Also, as shown in FIG. 2, when the second scope unit 2B is used the second scope unit 2B is connected to the fixed unit 3 through the amplification unit 60. Moreover, the fixed unit 3 and one model of the scope unit, for example the first scope unit 2A, are stored in a case main body 16 shown in FIG. 3, together with a storage section box 15 which will be described later. When the second scope unit 2B is used, the fixed unit 3, the second scope unit 2B, and also the amplification unit 60 are stored in the case main body 16 shown in FIG. 3, together with the storage section box 15 which will be described later.

Also, the first scope unit 2A has; a long and thin insertion section 4a which has flexibility, to be inserted at least into the space of the subject of examination, an intermediate connection section 4b, a universal cable 4c, and a base unit 4d. Here, the insertion section 4a comprises; a head section 4a1 arranged at the top most end position, with built in systems such as an observational optical system and an illumination optical system for observation, a curving section 4a2 which can be curved by remote operation, and a long and thin flexible tube section 4a3. Moreover the curving section 4a2 is provided between the head section 4a1 and the flexible tube section 4a3.

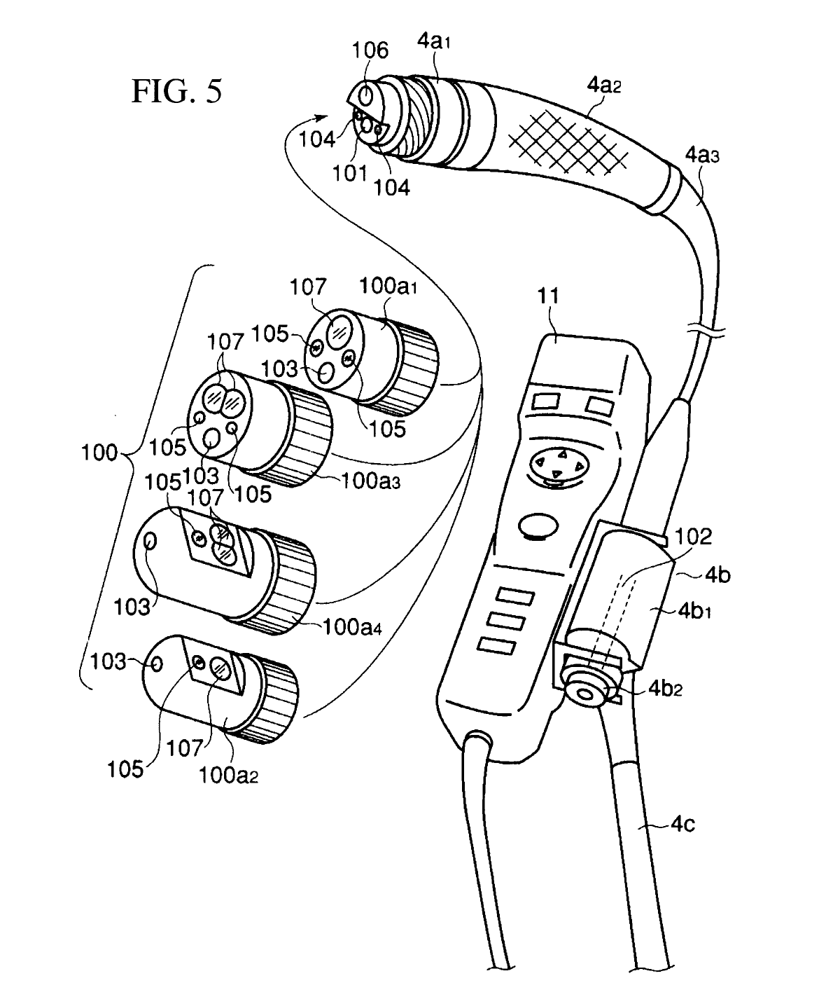

Furthermore, as shown in FIG. 5, two illumination windows 104 for the illumination optical system, an observation window 106 for the observation optical system, and a tip end opening 101 of an internal channel 102 (a path for pushing through a treatment tool) which is set inside the insertion section 4a, are respectively placed on the tip end face of the head section 4a1. Various types of optical adapters. 100 are appropriately selected and detachably installed as required on the tip end face of the head section 4a1. Such optical adapters 100 are, for example, optical adapters for normal observations of two dimensional images such as a direct sight adapter 100a1 and a side looking adapter 100a2, or binocular adapters which are used for stereoscopic measurement such as a direct sight binocular adapter 100a3 and a side looking binocular adapter 100a4.

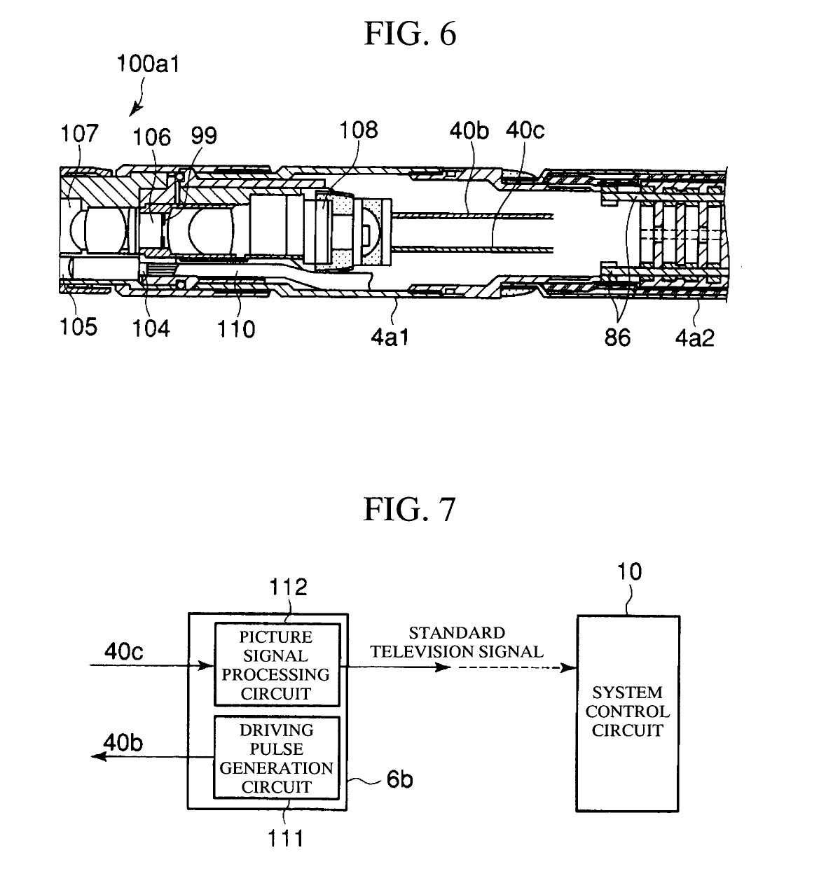

FIG. 6 is a cross section of a part in the vicinity of the head section 4a1 of the tip of the insertion section 4a with the direct sight adapter 100a1 attached to the tip end face of the head section 4a1. An adapter opening section 103 (refer to FIG. 5), adapter illumination windows 105, and an adapter observation window 107 are provided on the direct sight adapter 100a1, and are connected to the tip end opening 101, the illumination windows 104, and the observation window 106 on the head section 4a1 respectively.

As shown in FIG. 6, a light guide 110 which transmits illumination light to the illumination windows 104, signal conductors 40b and 40c which are connected to a solid-state image sensing device 108 such as a CCD set in the observational optical system, and a plurality of angle wires 86, four wires in the present embodiment, which control the curving of the curving section 4a2 in four directions, that is vertically and horizontally (up, down, left and right), are placed inside the insert section 4a. Furthermore, an aperture 99 is placed on the reverse face of the observation window 106. Moreover, the subject image incident from the observation window 106 is imaged on the solid-state image sensing device 108 which is placed on the focal plane, when adjusted by the aperture 99 for optimum exposure and depth of field. The solid-state image sensing device 108 is connected to a scope side connector 34, which will be described later, through the signal conductors 40b and 40c.

Also, in the present embodiment there are provided for example the two angle wires 86 for the vertical curve operations and the two angle wires 86 for the horizontal curve operations. Furthermore, the curving section 4a2 of the insertion section 4a is tractionally operated vertically by the two angle wires 86 for vertical curve operations, and horizontally by the two angle wires 86 for horizontal curve operations, respectively. As a result the curving section 4a2 of the insertion section 4a can be curved in four directions, namely vertically and horizontally, and in optional directions of combinations of these.

Also, as shown in FIG. 5, the tip end of the intermediate connection section 4b is connected to the base end of the flexible tube section 4a3 of the insertion section 4a. A grip section 4b1 which can be held by one hand of a user, is provided on the intermediate connection section 4b. On the back end of the grip section 4b1, a channel port section 4b2 and a junction with the tip end of the universal cable 4c are provided in parallel. Moreover, the light guide 110 which is extended from the insertion section 4a, the signal conductors 40b and 40c, and four of the angle wires 86 and the like are provided extendingly inside the universal cable 4c.

Also, the fixed unit 3 is provided with the storage box 15 (refer to FIG. 4) to store the insertion section 4a, or to store the insertion section 4a, the intermediate connection section 4b, the universal cable 4c, and the remote control 11. The storage box 15 is provided with a scope storage section 15a, a remote control storage section 15b, and a storage cover 15c. Furthermore, the insertion section 4a, or alternatively, the insertion section 4a, the intermediate connection section 4b, and the universal cable 4c are to be stored in the scope storage section 15a. The remote control 11 is to be stored in the remote control storage section 15b. Moreover, the scope storage section 15a and the remote control storage 15b are to be covered by the storage cover 15c.

Also, the base end of the universal cable 4c is connected to the base unit 4d. As shown in FIG. 1, an insertion section drive unit 5 and an electrical curve control unit 6a are built into the unit case 4d1 of the base unit 4d.

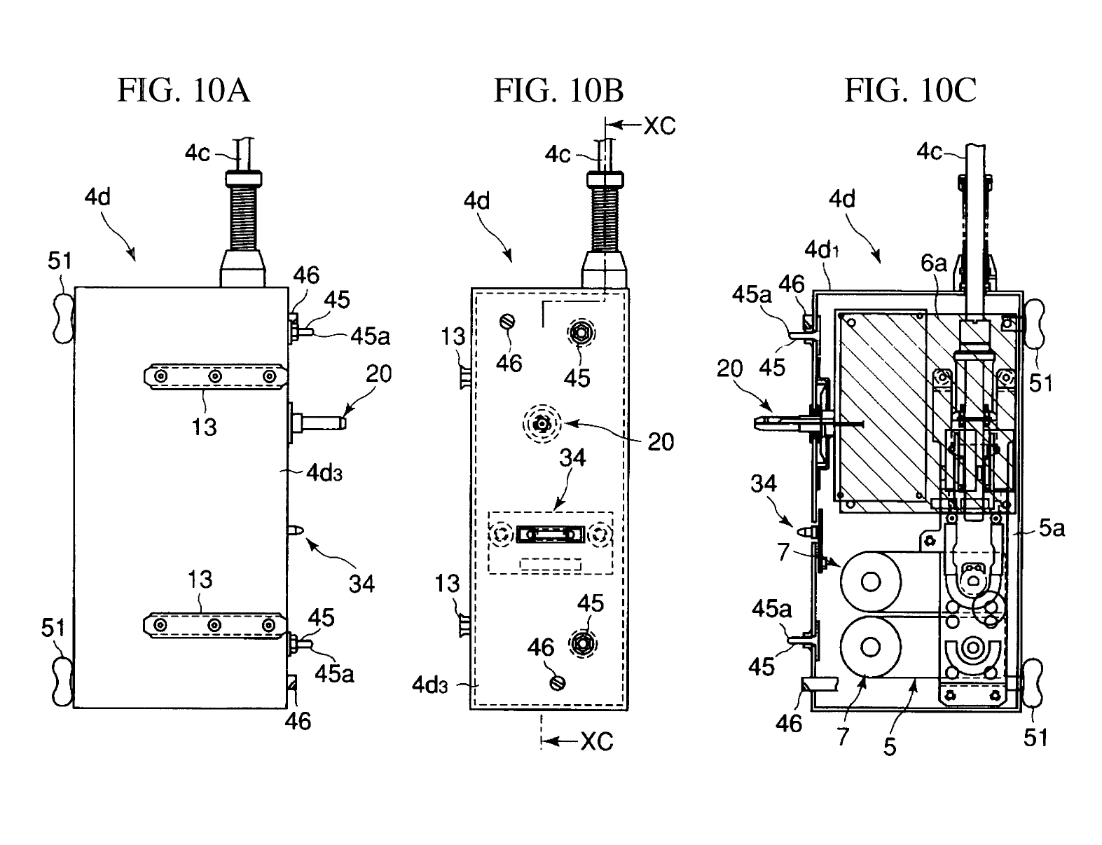

As shown in FIG. 10C, the insertion section drive unit 5 has a traction transmitting system unit 5a and two motor units 7 which correspond to the vertical curve operations and the horizontal curve operations respectively. The insertion section drive unit 5 drives the motor in accordance with motor drive signals from the electrical curve control unit 6a.

Also, driving pulses which drive the solid-state image sensing device 108 of the tip end head section 4a1 of the insertion section 4a and imager outputs which are the outputs of the solid-state image sensing device 108 are input to the insertion section drive unit 5.

The electrical curve control unit 6a comprises a ROM, being a storage device (not shown). Therefore, the electrical curve unit 6a maintains peculiar information of the first scope unit 2A, such as an electrical curve control program of the first scope unit 2A, the length of the insertion section 4a, the diameter of the insertion section 4a, and a serial number. The information contained in the ROM of the electrical curve control unit 6a is used for the control of the electrical curving section 4a2 and the control of the amplification unit 60, which will be described later, of the fixed unit 3.

Also, driving pulses which drive the solid-state image sensing device 108 of the tip end head section 4a1 of the insertion section 4a and the imager outputs which are the outputs of the solid-state image sensing device 108, are input to the electrical curve control unit 6a.

Moreover, a power unit 8, a light source unit 9, a system control circuit 10, a lamp lighting circuit (not shown), a circuit intermediate substrate, and a CCU control unit 6b are built into the unit case 3a of the fixed unit 3. The CCU control unit 6b has a built in control circuit comprising a camera control unit (CCU) which controls the solid-state image sensing device 108 and converts signals of the taken image into standard television signals.

Furthermore, the remote control 11 which controls the endoscope apparatus 1 and a monitor 12 for image display, are arranged outside of the fixed unit 3 and are provided to be able to connect to the system control circuit 10.

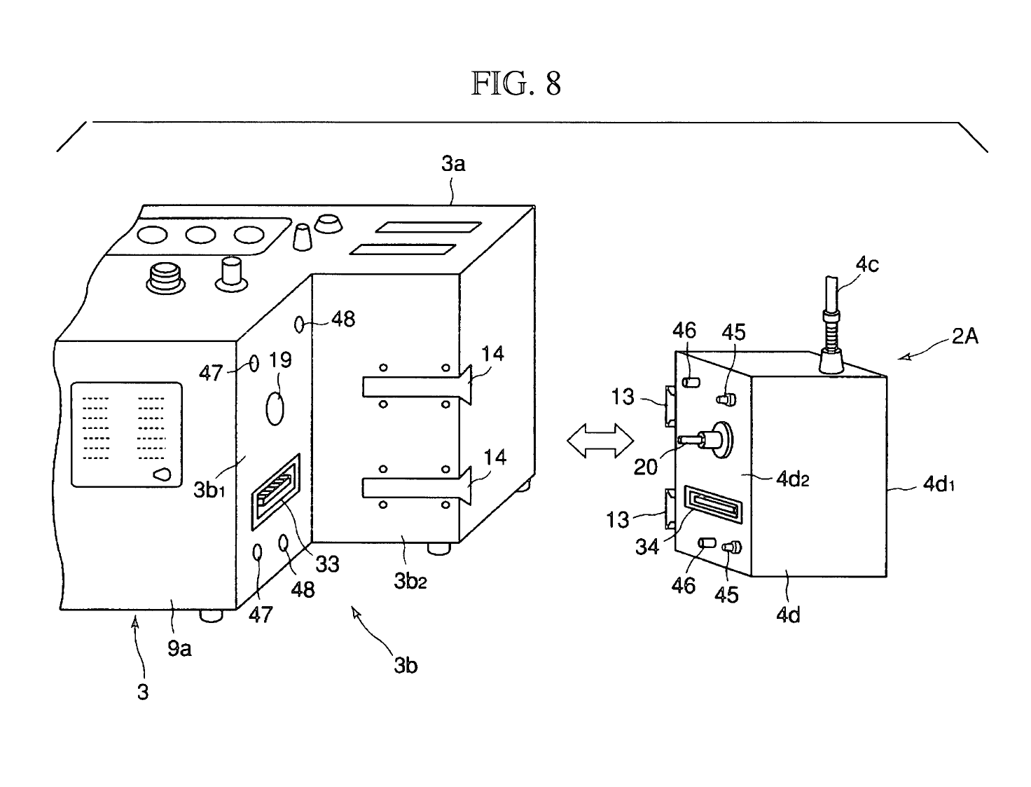

Also, as shown in FIG. 8, a socket shaped junction 3b which detachably connects the base unit 4b of the scope unit 2, is formed on the unit case 3a of the fixed unit 3. A connection surface 3b1 which touches with an end panel 4d2 (a box body panel) of the unit case 4d1, and a scope unit contact surface 3b2 which contacts with the side panel 4d3 (refer to FIG. 10B) of the unit case 4d1 in the base unit 4d of the first scope unit 2A, are provided on the junction 3b. Here, the connection surface 3b1 is formed by a lamp housing 9a of the light source unit 9 in the fixed unit 3.

Also, as shown in FIG. 10A, two protruding resin slider members 13, each of which extends in the substantially horizontal direction, are vertically arranged on the side panel 4d3 of the unit case 4d1 of the base unit 4d. The slider members 13 guide the movement of the base unit 4d when it is connected to the fixed unit 3. Metallic guide rails 14 are provided on the fixed unit 3 to guide the movement of the slider components 13.

Also, as shown in FIG. 1, an optical connector unit 17 for optical connections and an electrical connector unit 18 for electrical connections are provided in the connection section between the connection surface 3b1 of the fixed unit 3 and the end panel 4d2 of the base unit 4d in the scope unit 2. The connection units 17 and 18 are mechanical connection interface units that detachably connect between the base unit 4d of the scope unit 2 and the fixed unit 3 to make them function as an endoscope.

The scope side electrical connector 34 which is attached to the scope unit 2, and an fixed unit side electrical connector 33 which is attached to the fixed unit 3 are provided on the electrical connector unit 18. Moreover, the scope unit 2 and the fixed unit 3 are connected electrically by detachably connecting the scope side electrical connector 34 and the fixed unit side electrical connector 33.

A harness 40 of the scope unit 2 includes a plurality of signal conductors, for example a signal conductor for driving signals that are output from a driving pulse generation circuit 111 inside of the CCU control unit 6b which will be described later, a signal conductor for imager outputs which are output from the solid-state image sensing device 108, a power conductor for the solid-state image sensing device 108 on the tip end of the endoscope, a signal conductor for communication signals to control the electrical curve control unit 6a, and power conductors for the electrical curve control unit 6a and motors. These signal conductors are connected to the CCU control unit 6b and the system control circuit 10 through a harness 37 which is connected to the fixed unit side electrical connector 33.

As shown in FIG. 7, the driving pulse generation circuit 111 and a picture signal processing circuit 112 are built into the CCU control unit 6b. The driving signals which emits from the driving pulse generation circuit 111 are relayed by the electrical curve control unit 6a and the insertion section drive unit 5, and transmitted to the tip end by a driving pulse conductor 40b which is inserted through inside the endoscope. Then the solid-state image sensing device 108 in the tip end head section 4a1 is driven by these driving signals.

Also, functions to perform the following processes are provided in the system control circuit 10.

(1) Control the overall endoscope apparatus 1.

(2) Create and transmit the communication command with respect to the CCU control unit 6b accompanied by the input of picture manipulation signals such as for image zoom, from the remote control 11.

(3) Create the communication command with respect to the electrical curve control unit 6a accompanied by the input of electrical curve manipulation signals from the remote control 11, and transmit via the signal conductor 40a.

(4) The electrical curve control unit 6a drives the motor 7 in accordance with the input commands.

(5) In addition, display a menu (not shown), and control the overall endoscope apparatus 1 in accordance with this.

Also, in addition to the signal conductors mentioned above, a connection detection conductor which detects whether or not the scope unit 2 is connected normally is included in the scope side electrical connector 34 and the fixed unit side electrical connector 33. The connection detection conductor is pulled up in the system control circuit 10 and grounded in the scope unit 2. Therefore, the system control circuit 10 is able to detect when the scope unit 2 is connected.

Moreover, between the fixed unit 3 and the scope unit 2, information such as the curving angle of the electrical curving section 4a2, existence/nonexistence of error occurrences, and the length of the insertion section 4a is exchanged through serial communication such as RS-232C. The communication signals are communicated through the signal conductor 40a.

Furthermore, a light guide connector (hereafter referred to as an LG connector) 20 which is attached to the scope unit 2, and a light source side optical connector 19 which is attached to the fixed unit 3, are provided for the optical connector unit 17. The LG connector 20 is a fixed connector which is attached to the fixed unit 3 relatively strongly with good location accuracy. The light source side optical connector 19 is a movable connector which is attached with looseness (play) so that it can slightly move with respect to the base unit 4d.

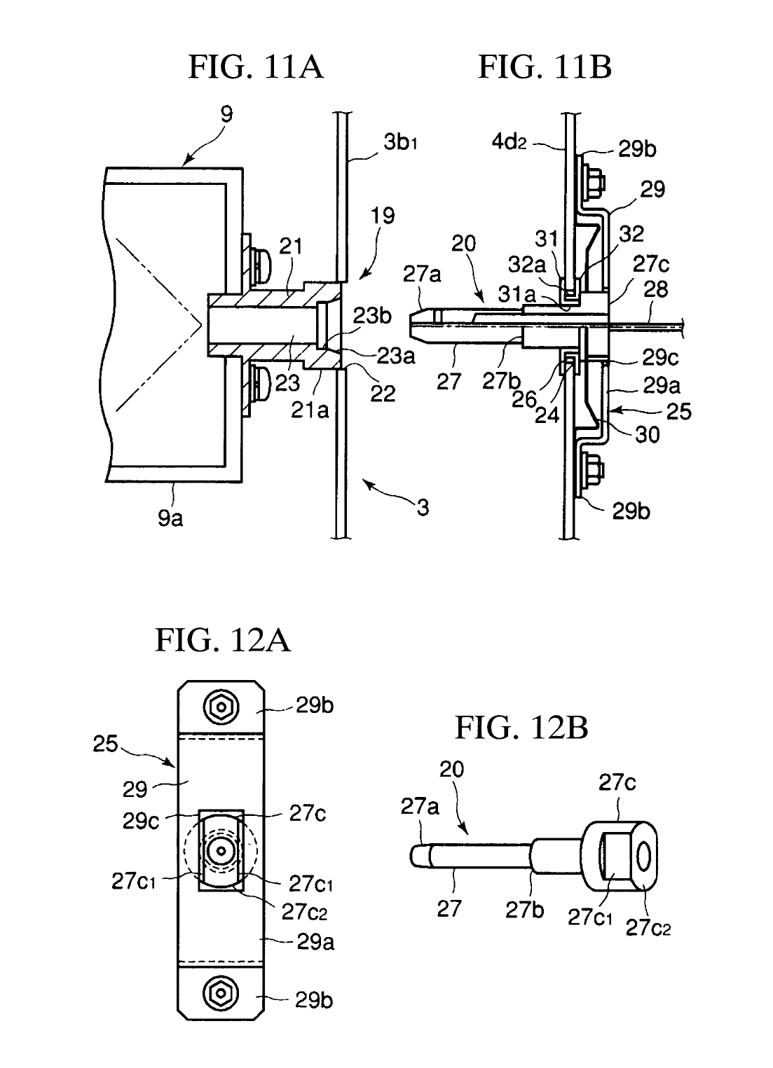

FIG. 11A shows the attaching state of the light source side optical connector 19. Here, an approximate tubular connector block (receiver) 21 which fits the LG connector 20 is provided in the light source side optical connector 19. The base end of the connector block 21 is screwed and fixed to the lamp housing 9a of the light source unit 9 in the fixed unit 3.

Furthermore, a mouthpiece 21a with a large diameter is formed at the tip end of the connector block 21. The mouthpiece 21a is installed in a connector installation hole 22 which is formed in a connection surface 3b1 of the fixed unit 3.

Also, an LG connector insertion hole 23 for inserting the LG connector 20 is formed in the tube of the connector block 21. A mouth piece taper section (taper shaped interfit hole) 23a with an inside diameter which gradually widens toward the outside so as to easily insert the LG connector 20 into the entrance of the connector block 21, is formed at the tip end of the LG connector insertion hole 23. Moreover, a step section 23b which becomes a dead end surface when inserting the LG connector 20, is provided at rear end position of the mouthpiece taper section 23a in the mouthpiece section 21a.

FIG. 11B shows the attaching state of the LG connector 20. Here, an LG connector attachment hole 24 is formed in the end panel 4d2 of the unit case 4d1 in the base unit 4d of the scope unit 2. The LG connector 20 is inserted into the attachment hole 24, and in such a state the LG connector 20 is supported by an LG connector supporting section 25. A space section 26 which allows loose movement between both connectors when the LG connector 20 is attached/detached with respect to the light source side optical connector 19 of the fixed unit 3, is provided on the LG connector supporting section 25.

Also, an approximate shaft shaped LG connector main body 27 is provided on the LG connector 20. The base end of a light guide 28 which is extended from the universal cable 4c is connected to the center of the shaft of the LG connector main body 27.

Furthermore, a taper shaped taper section 27a is formed on the tip end of the LG connector main body 27 so as to easily insert into the connector block 21 on the fixed unit 3 side. A step section 27b is provided midway along the LG connector main body 27 to correspond to the dead end surface of the step section 23b of the connector block 21.

Also, as shown in FIG. 12B, a large diameter shaft section 27c is formed at the root side end of the LG connector main body 27. A D-cut section 27c1 with notches on opposite sides, is formed on the rear end of the large diameter shaft section 27c.

Moreover, as shown in FIG. 11B, a connector receiver 29, a plate spring member 30, and two connector receiver rings, which are a first connector receiver ring 31 and a second connector receiver ring 32, are provided on the LG connector supporting section 25. Here, a small diameter cylinder section 31a is provided protrudingly in the inner circumference of the first connector receiver ring 31. The inner circumference surface of the small diameter cylinder section 31a in the first connector receiver ring 31 is fitted with the outer circumference surface of the LG connector 20.

Also, a male screw section is formed on the outer circumference surface of the small diameter cylinder section 31a of the first connector receiver ring 31. Furthermore, a screw joining cylinder section 32a having a tapped hole which is screwed and joined, is formed in the inner circumference of the second connector receiver ring 32. The outer diameter of the screw joining cylinder section 32a is set to be smaller than the inner diameter of the LG connector attachment hole 24.

Moreover, the first connector receiver ring 31 and the second connector receiver ring 32 are fitted from the opposite faces of the end panel 4d2 of the unit case 4d1. The first connector receiver ring 31 and the second connector receiver ring 32 are screwed and combined to each other by screw threads which are provided on both rings. In this state, the space section 26 is formed by the space between the screw joining cylinder section 32a of the second connector receiver ring 32 and the LG connector attachment hole 24. Then, when the combined first and second connector receiver rings 31 and 32 are attached to the end panel 4d2 of the unit case 4d1, the combined first and second connector receiver rings 31 and 32 can move freely in the direction orthogonal to the axial direction of the LG connector 20 within the range of the space section 26. As a result, the LG connector 20 can move freely in the direction orthogonal to the axial direction of the LG connector 20 within the range of the space section 26.

Moreover, as shown in FIG. 11B, approximate L-shaped leg sections 29b are bent and formed on the opposite ends of an approximate tabular base plate 29a of the connector receiver 29. Furthermore, as shown in FIG. 12A, a rectangular hole 29c is formed at the approximately central position of the base plate 29a.

Also, the plate spring member 30 is placed inside of the connector receiver 29. A rectangular hole is provided in the plate spring member 30. A shaft section 27c2 between the D-cut sections 27c1 on opposite sides of the root side end of the LG connector 20, is inserted into the rectangular hole. Moreover, the shaft section 27c2 between the D-cut sections 27c1 on opposite sides of the root side end of the LG connector 20, is inserted through the rectangular hole 29c of the connector receiver 29.

As shown in FIG. 12A, the rectangular hole 29c is set to be slightly larger than the cross section of the shaft section 27c2 between the D-cut sections 27c1 on opposite sides of the root side end of the LG connector 20. The rectangular hole 29c is to not limit the movement in the direction orthogonal to the axial direction of the LG connector 20 within the play of the LG connector 20, that is, the range of the space section 26. Furthermore, the turning angle of the LG connector 20 is controlled by an engaging section of the shaft section 27c2 between the D-cut sections 27c1 on opposite sides of the large diameter shaft section 27c of the LG connector main body 27 and the rectangular hole 29c. Therefore, the light guide 28 does not get damaged by twisting.

Moreover, as shown in FIG. 11B, the leg sections 29b of the connector receiver 29 are fastened together with the plate spring member 30 by lock nuts to the end panel 4d2 of the unit case 4d1. At this time, by sufficiently reinforcing the connector receiver 29, the LG connector 20 can be limited to move within the elastic area of the plate spring member 30 when the LG connector 20 is in contact with other parts. Accordingly, the LG connector 20 is kept from pressing and damaging the parts inside.

Furthermore, when the light source side optical connector 19 and the LG connector 20 of the optical connector unit 17 are connected, the LG connector 20 of the scope unit 2 is inserted into the tube of the connector block 21 of the fixed unit 3. At this time, the connector block 21 is attached in the connector installation hole 22 of the fixed unit 3 with good location accuracy and fixed relatively strongly. On the other hand, the LG connector 20 is supported by the LG connector supporting section 25 with looseness (play) so that it can slightly move with respect to the end panel 4d2 of the unit case 4d1 of the scope unit 2. Accordingly, the loose movement between both connectors when attaching/detaching the LG connector 20 with respect to the light source side optical connector 19 of the fixed unit 3 can be absorbed by the free movement of the LG connector 20 in the direction orthogonal to the axial direction of the LG connector 20 within the range of the space section 26. Then, in the state where the taper section 27a at the tip of the LG connector 20 is in contact with the mouthpiece taper section 23a of the connector block 21 and guided along the mouthpiece taper section 23a, the LG connector 20 is inserted into the inside of the LG connector insertion hole 23. Accordingly, when the light source side optical connector 19 of the fixed unit 3 and the LG connector 20 are connected, an axial matching device which matches the axes thereof is formed.

Moreover, when the LG connector 20 is fitted into the connector block 21 of the light source side optical connector 19, the position of the connector block 21 and a lamp (not shown) are adjusted so that the optical axis of the lamp (not shown) in the light source unit 9 and the end face of the light guide 28 of the LG connector 20 become coaxial. Accordingly, the illumination light of the lamp (not shown) in the light source unit 9 is focused on the connector end face of the LG connector 20.

Furthermore, when the endoscope apparatus 1 of the present embodiment is used, the illumination light supplied by the light source unit 9 is transmitted through the LG connector 20 to the insertion section light guide 28. Then, the illumination light irradiates the front subject by the illumination optical system 104 placed in the tip end head section 4a1. The subject image illuminated at this time is adjusted via the objective optical system and the observation window 106 shown in FIG. 5 and FIG. 6, by the aperture 99 for optimum exposure and depth of field, then imaged on the solid-state image sensing device 108 which is placed on the focal plane.

The imager outputs (image signals) which are output from the solid-state image sensing device 108 are sent to the scope side connector 34 through the signal conductor 40c and supplied to the CCU control unit 6b through the fixed unit side electrical connector 33. Furthermore, in the CCU control unit 6b, the image signals are input into the picture signal processing circuit 112 and converted into standard television signals.

Moreover, the standard television signals are further input into the system control circuit 10 and A/D converted. The A/D converted picture signals are recorded into a PC card for image recording which is inserted into a PC card slot of a record unit (not shown), accompanied by pressing an image recording button (not shown) of the remote control 11. Moreover, the A/D converted picture signals are D/A converted and output to the monitor 12.

FIG. 14A to FIG. 14C show an fixed unit side electrical connector 33 which is the fixed connector attached to the connection surface 3b1 of the fixed unit 3 in the standard position. The electrical connector main body 35 of the fixed unit side electrical connector 33 is mounted on the substrate 36. The one end of the harness 37 is connected to the substrate 36. The other end of the harness 37 is connected to the system control circuit 10 and the CCU control unit 6b in the fixed unit 3.

Furthermore, as shown in FIG. 14B, positioning connector hollows 35a are respectively provided at opposite ends of the electrical connector main body 35. Also, as shown in FIG. 14C, the substrate 36 having the electrical connector 33 mounted thereon, is fixed on the end panel 4d2 of the unit case 4d1 with good location accuracy.

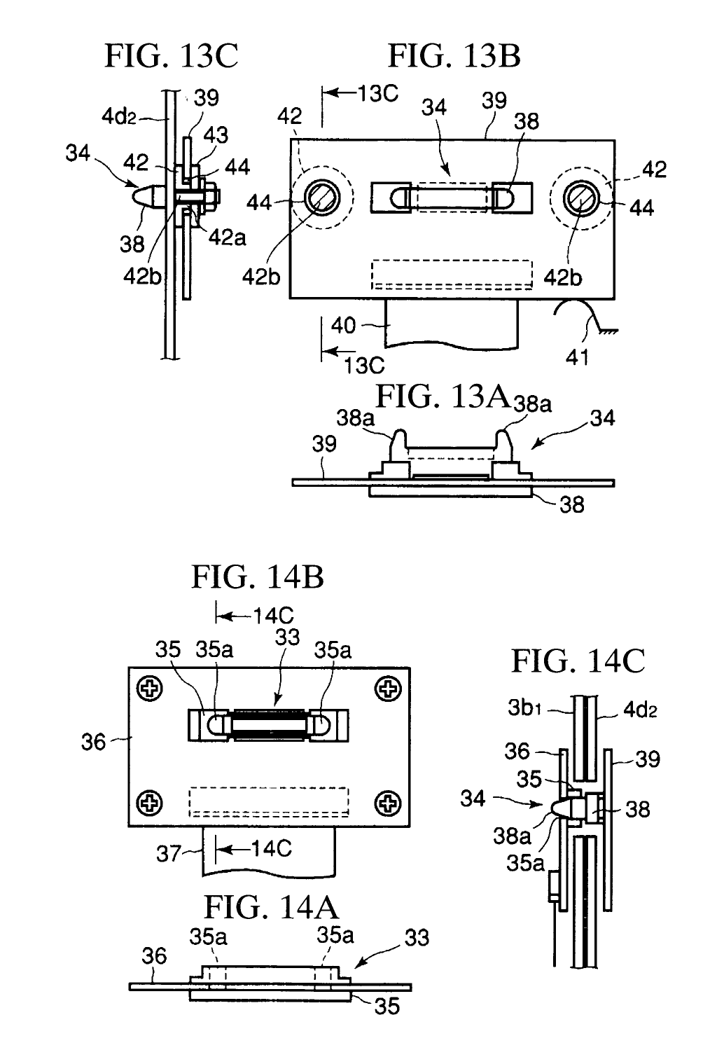

FIGS. 13A to C shows an scope side electrical connector 34 which is a movable connector attached on the end panel 4d2 of the unit case 4d1 of the scope unit 2. The electrical connector main body 38 of the scope side electrical connector 34 is mounted on a substrate 39. One end of the harness 40 is connected to the substrate 39. The other ends of the harnesses 40a, 40b, and 40c are connected to the electrical curve control unit 6a and the insertion section drive unit 5 in the scope unit 2.

Also, as shown in FIG. 13A, positioning connector projections 38a are respectively provided on the opposite ends of the electrical connector main body 38. The connector projections 38a are arranged in positions corresponding to the two connector hollows 35a of the fixed unit side electrical connector 33.

Furthermore, as shown in FIG. 13C, the substrate 39 having the electrical connector 34 mounted thereon, is fixed to the end panel 4d2 of the unit case 4d1 through two approximate ring shaped spacer rings, which are a first spacer ring 42 and a second spacer ring 43. Here, a small diameter cylinder section 42a is provided protrudingly in the inner circumference of the first spacer ring 42. The inner circumference surface of the small diameter cylinder section 42a in the first spacer ring 42 is fitted with the outer circumference surface of the spindle 42b of the substrate 39. The outer diameter of the spindle 42b is set to be smaller than the inner diameter of the substrate attachment hole 44 which is formed in the substrate 39. Moreover, the substrate 39 can move freely in the direction orthogonal to the axial direction of the spindle 42b within the range of the space between the spindle 42b and the substrate attachment hole 44 of the substrate 39. Accordingly a space section which allows loose movement between both connectors when the fixed unit side electrical connector 33 and the scope side electrical connector 34 are attached/detached, is formed. As shown in FIG. 13B, the substrate 39 may be reinforced by a spring member 41 so that the substrate 39 will not loosely move due to vibration or the like.

When the fixed unit side electrical connector 33 and the scope side electrical connector 34 are connected, axial matching for determining the position of the connector projections 38a on the opposite ends of the electrical connector main body 38 is performed to adjust with the position of the connector hollows 35a, at the time when the connector projections 38a on the opposite ends of the electrical connector main body 38 are respectively fitted with the two connector hollows 35a of the fixed unit side electrical connector 33.

Furthermore, when the fixed unit side electrical connector 33 and the scope side electrical connector 34 are connected, since the substrate 39 of the scope side electrical connector 34 moves within the range of the space between the spindle 42b and the substrate attachment hole 44 of the substrate 39, the connector projections 38a of the electrical connector main body 38 are inserted and smoothly connected into the connector hollows 35a of the electrical connector main body 35.

Also, as shown in FIG. 8, a guide pin 45 and a lock member 46 are respectively placed on the top end of the end panel 4d2 of the base unit 4d of the scope unit 2. Similarly, the guide pin 45 and the lock member 46 are respectively placed on the bottom end of the end panel 4d2 of the base unit 4d.

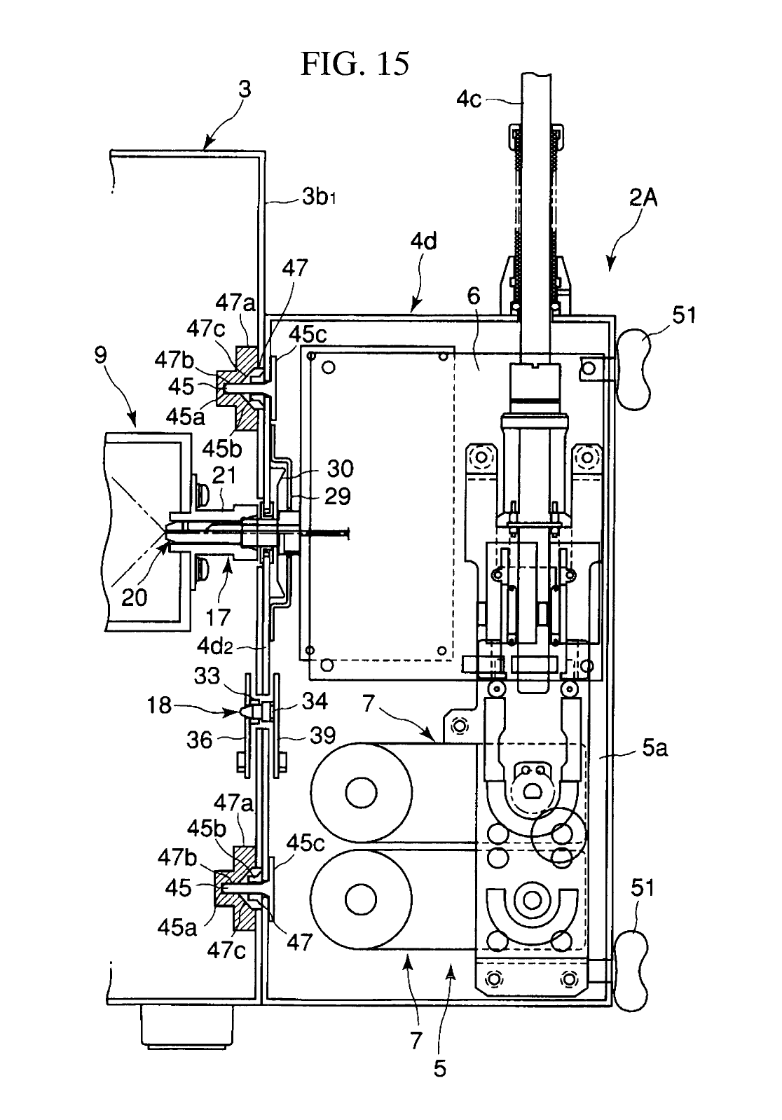

As shown in FIG. 15, a flange 45c is formed on the base end of the shaft member of the guide pin 45. The guide pin 45 pierces from the reverse face of the end panel 4d2 of the unit case 4d1, and is fixed by a nut 45b. Accordingly, the assembling becomes easier with good location accuracy. At this time, the guide pins 45 are attached to the end panel 4d2 of the unit case 4d1 with good location accuracy. Furthermore, a taper shaped taper section 45a is formed at the tip of the shaft member of the guide pin 45 so as to fit easily.

Also, as shown in FIG. 15, guide pin receivers 47 are attached in the position corresponding to the guide pins 45 of the base unit 4d on the connection surface 3b1 of the fixed unit 3 with good location accuracy. A pin insertion hole 47b into which the guide pin 45 is inserted, is formed in the main body 47a of the guide pin receiver 47. A tapered surface 47c is formed at the entrance of the insertion hole 47b so as to fit easily.

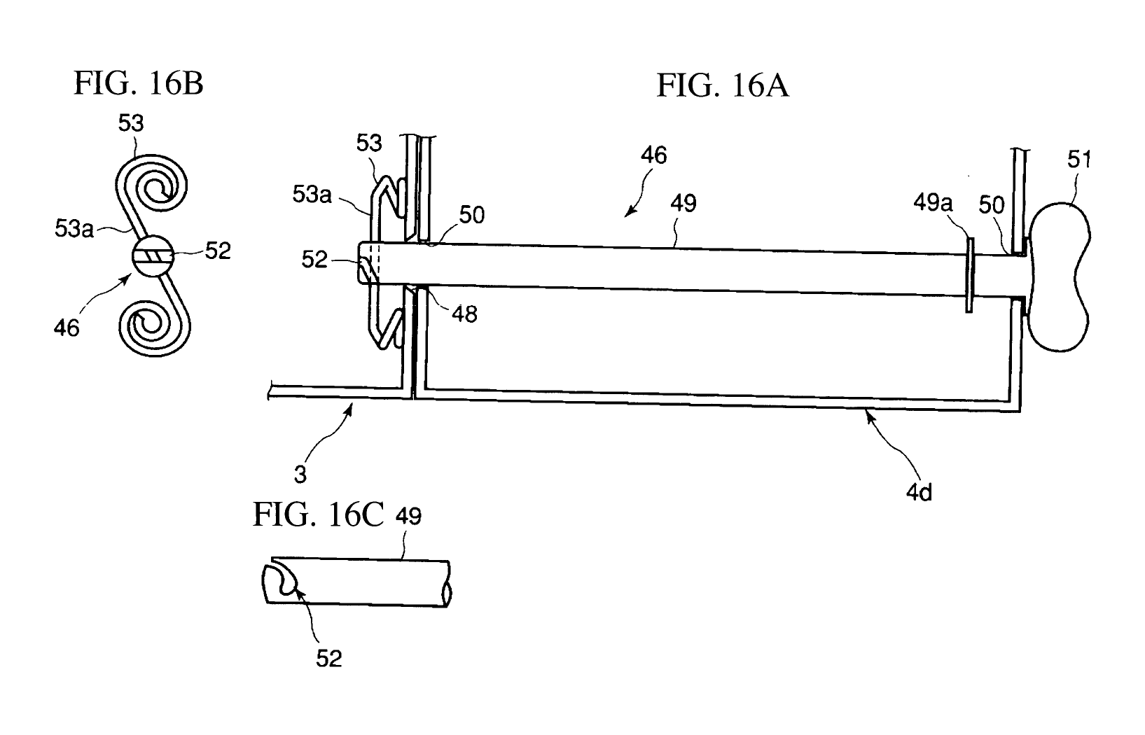

Moreover, FIG. 16A shows a lock member 46 which fastens and fixes when two box bodies of the fixed unit 3 and the base unit 4d of the scope unit 2 are connected. A shaft 49 which pierces the base unit 4d and is extended to the opposite sides of the shaft piercing hole 50, is provided on the lock member 46. A knob 51 which is arranged outside of the base unit 4d, is provided on the base end of the shaft 49. As shown in FIG. 16C, an approximate spiral locking groove 52 is formed at the tip end of the shaft 49. An E ring attaching groove is formed partway along the shaft 49. The shaft 49 is kept from being detached from the scope unit 2 by means of an E ring 49a provided in the E ring attaching groove.

Furthermore, a lock hole 48 is formed in the position corresponding to the lock member 46 of the scope unit 2 in the box body panel on the connection surface 3b1 of the fixed unit 3. A spring member 53 for locking the lock member 46 is fixed on the reverse face of the connection surface 3b1 around the lock hole 48. As shown in FIG. 16B, a straight locking section 53a is formed in the spring member 53. The locking groove 52 at the tip of the shaft 49 of the lock member 46 is to be unlockably locked to the locking section 53a.

In the system of the endoscope apparatus 1 of the present embodiment, a second scope unit 2B may be used instead of the first scope unit 2A. As shown in FIG. 2, when the second scope unit 2B is used, an amplification unit 60 is used. The second scope unit 2B is connected to the fixed unit 3 through the amplification unit 60.

Moreover, similarly to the first scope unit 2A, an insertion section 4a is provided for the second scope unit 2B. The insertion section 4a has a head section 4a1, a curving section 4a2 and a flexible tube section 4a3. The insertion section 4a of the second scope unit 2B has the insertion length longer than that of the insertion section 4a of the first scope unit 2A, for example as long as 10 m or more. Furthermore, the universal cable 4c of the first scope unit 2A is not provided for the second scope unit 2B. In the second scope unit 2B, the end on the handle side of the insertion section 4a is directly connected to the base unit 4d. Accordingly, using the second scope unit 2B, the whole length of the insertion section 4a may be inserted into the specimen.

Similarly to the base unit 4d of the first scope unit 2A, the base unit 4dof the second scope unit 2B comprises; an insertion section drive unit 5, a motor unit 7, an electrical curve control unit 6a, an optical connector unit 17, and a power connector unit 18. Furthermore, similarly to the base unit 4d of the first scope unit 2A, it has a unit case 4d1, an end panel 4d2, an LG connector 20, a scope side electrical connector 34, slider members 13, guide pins 45, and lock members 46 of the same structure.

Furthermore, as shown in FIG. 4, an approximate rectangular box type unit case 60a is provided for the amplification unit 60. A base unit assembling surface 61 which is assembled with the base unit 4d on the front face and a fixed unit assembling surface 62 which is assembled with the connection surface 3b1 of the fixed unit 3 on the rear face, are respectively formed in the unit case 60a.

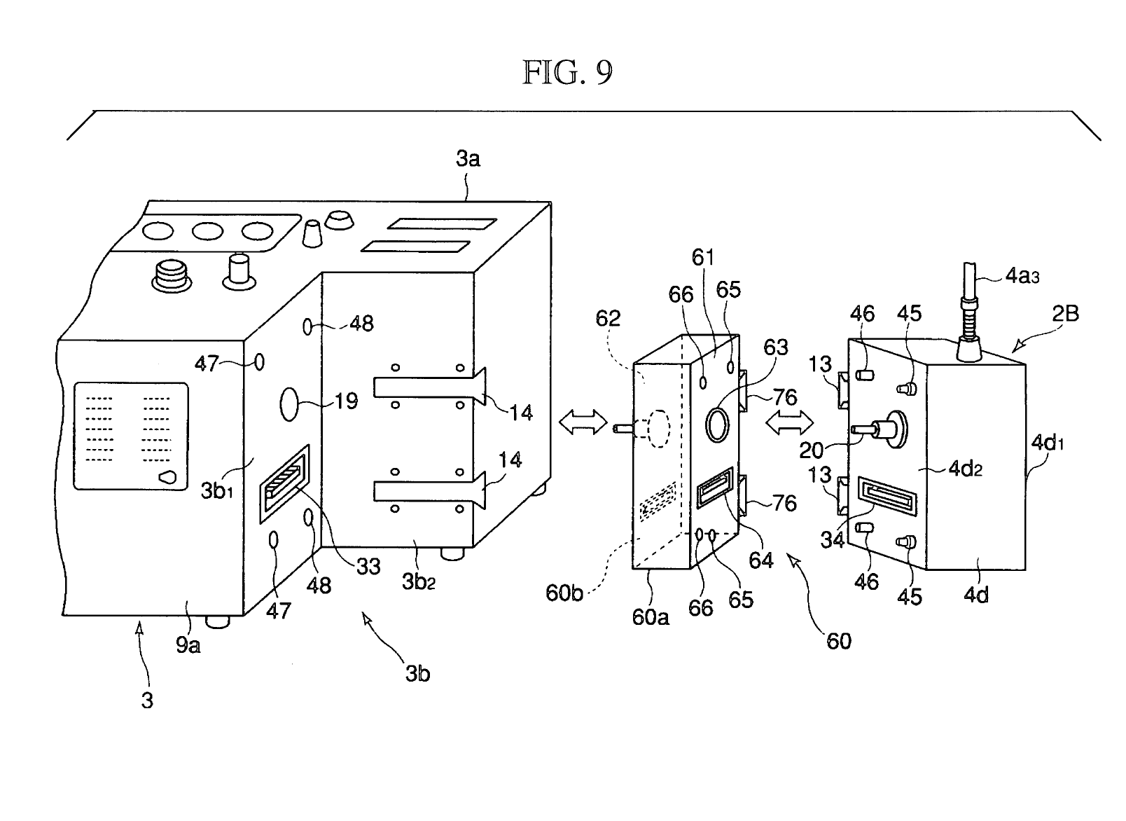

The base unit assembling surface 61 has approximately the same structure as that of the connection surface 3b1 of the fixed unit 3. That is, as shown in FIG. 9, a female type amplification side optical connector 63, a female type amplification side electrical connector 64, and an amplification side guide pin receiver 66, having the same structure as the light source side optical connector 19, the fixed unit side electrical connector 33, and the guide pin receiver 47 of the fixed unit 3, are respectively provided on the base unit assembling surface 61. A lock coupler mechanism 65 which will be described later is provided in the position corresponding to the lock hole 48 of the fixed unit 3 on the base unit assembling surface 61.

Moreover, the fixed unit assembling surface 62 of the amplification unit 60 has approximately the same structure as that of the end panel 4d2 of the base unit 4d. That is, as shown in FIG. 4, a male type amplification side optical connector 72, a male type amplification side electrical connector 73, and an amplification side guide pin 71 having a similar structure to the male type LG connector 20, the scope side electrical connector 34 and the guide pin 45 on the end panel 4d2 of the base unit 4d, are respectively provided on the fixed unit assembling surface 62. A lock coupler mechanism 65 which will be described later is provided in the position corresponding to the lock member 46 of the base unit 4d on the fixed unit assembling surface 62.

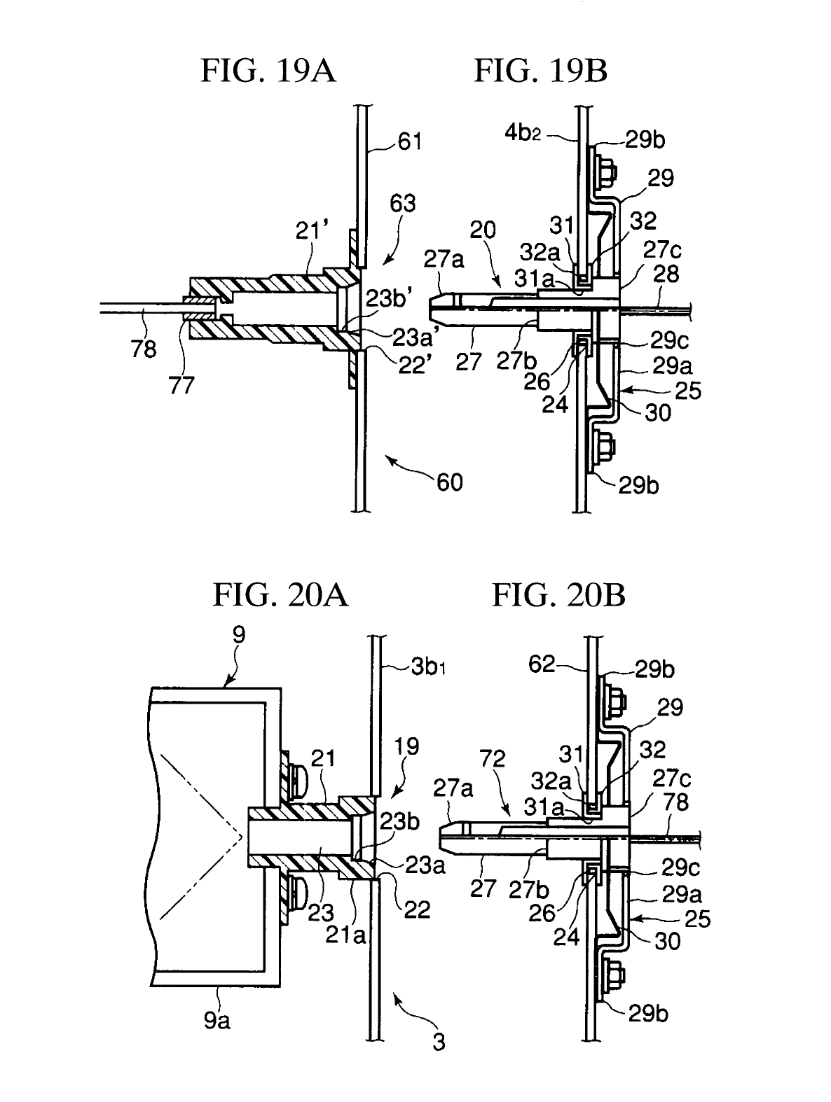

FIG. 19A shows the attaching state of the amplification side optical connector 63. Here, a fixed connector installation hole 22′ is formed in the base unit assembling surface 61 of the amplification unit 60. An approximate tubular connector block (receiver) 21′ into which the LG connector 20 is inserted, is provided on the amplification side optical connector 63. One end of the connector block 21′ is attached in the fixed connector installation hole 22′ with good location accuracy and fixed relatively strongly. The LG connector 20 of the base unit 4d is to be detachably connected to the amplification side optical connector 63.

As shown in FIG. 19B, a space section 26 which allows loose movement between both connectors when the abovementioned LG connector 20 is attached/detached, is provided on the LG connector 20. Accordingly, the loose movement between both connectors when attaching/detaching the LG connector 20 with respect to the amplification side optical connector 63 can be absorbed by the free movement of the LG connector 20 in the direction orthogonal to the axial direction of the LG connector 20 within the range of the space section 26 of the connector supporting section 25. Then, in the state where the taper section 27a at the tip of the LG connector 20 is in contact with the mouthpiece taper section 23a′ of the connector block 21′ and guided along the mouthpiece taper section 23a′, the LG connector 20 is inserted into the inside of the LG connector insertion hole 23′. Accordingly, when the amplification side optical connector 63 and the LG connector 20 are connected, an axial matching device which matches the axes thereof is formed.

Furthermore, the one end of the relay LG bundle 78 is connected to the other end of the connector block 21′ of the amplification side optical connector 63. A mouthpiece 77 is inserted at the end of the relay LG bundle 78. The one end of the relay LG bundle 78 is screwed and fixed, or adhered and fixed through the mouthpiece 77.

As shown in FIG. 20B, the other end of the relay LG bundle 78 is connected to the amplification side optical connector 72. The other end of the relay LG bundle 78 has the same shape as the end of the light guide 28. The structure of the amplification side optical connector 72 is the same as that of the LG connector 20. Therefore, the same reference symbols are used for components the same as those for the LG connector 20, and description thereof is omitted here. The amplification side optical connector 72 is to be detachably connected to the light source side optical connector 19 of the fixed unit 3 shown in FIG. 20A.

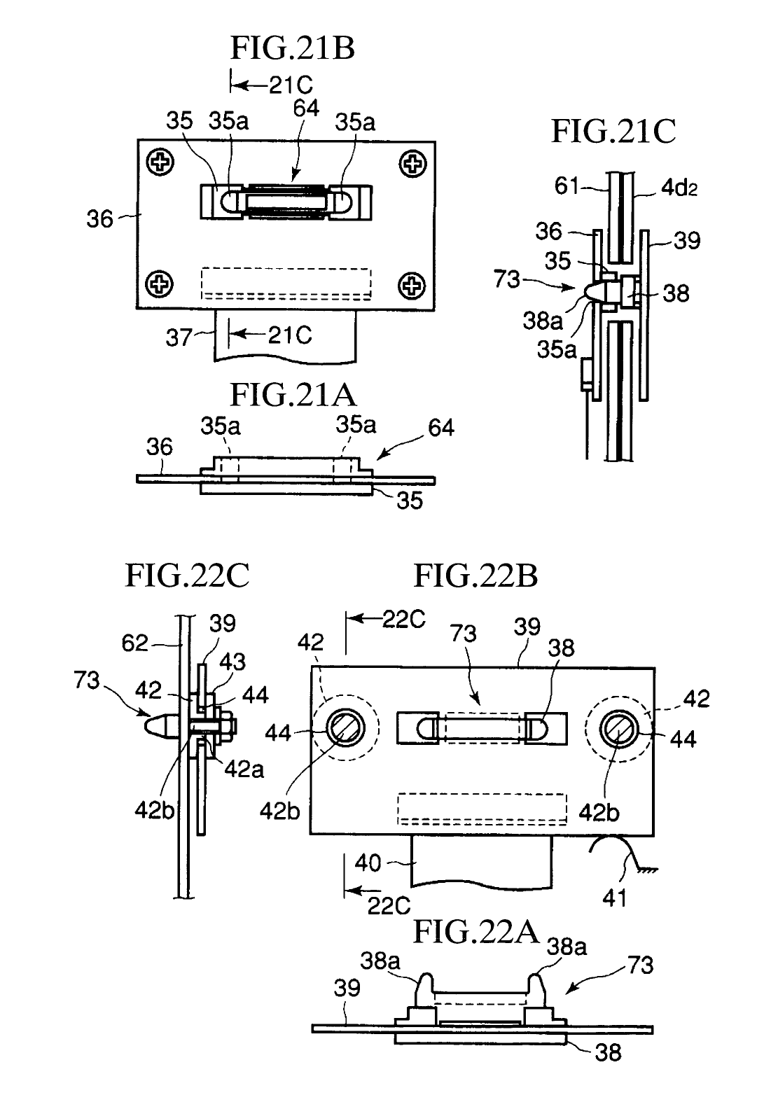

FIGS. 21A to C show an amplification side electrical connector 64 which is a standard position fixed connector attached to the base unit assembling surface 61 of the amplification unit 60. The amplification side electrical connector 64 has the same structure as that of the fixed unit side electrical connector 33 of the fixed unit 3. Therefore, the same reference symbols are used for components the same as those for the fixed unit side electrical connector 33 and description thereof is omitted here. The scope side electrical connector 34 of the base unit 4d is to be detachably connected to the amplification side electrical connector 64.

FIGS. 22A to C show an amplification side electrical connector 73 which is attached to the fixed unit assembling surface 62 of the amplification unit 60. The amplification side electrical connector 73 has the same structure as that of the scope side electrical connector 34 of the base unit 4d. Therefore, the same reference symbols are used for components the same as those for the scope side electrical connector 34 and description thereof is omitted here. The amplification side electrical connector 73 is to be detachably connected to the fixed unit side electrical connector 33 of the fixed unit 3.

Moreover, as shown in FIG. 2, the amplification unit 60 comprises an amplification circuit section 74. The amplification circuit section 74 has a function to amplify the drive wave form of the CCU control unit 6b, and to form necessary wave forms for operating the solid-state image sensing device 108 such as the CCD in the remote position, when the second scope unit 2B and the fixed unit 3 are assembled via the amplification unit 60.

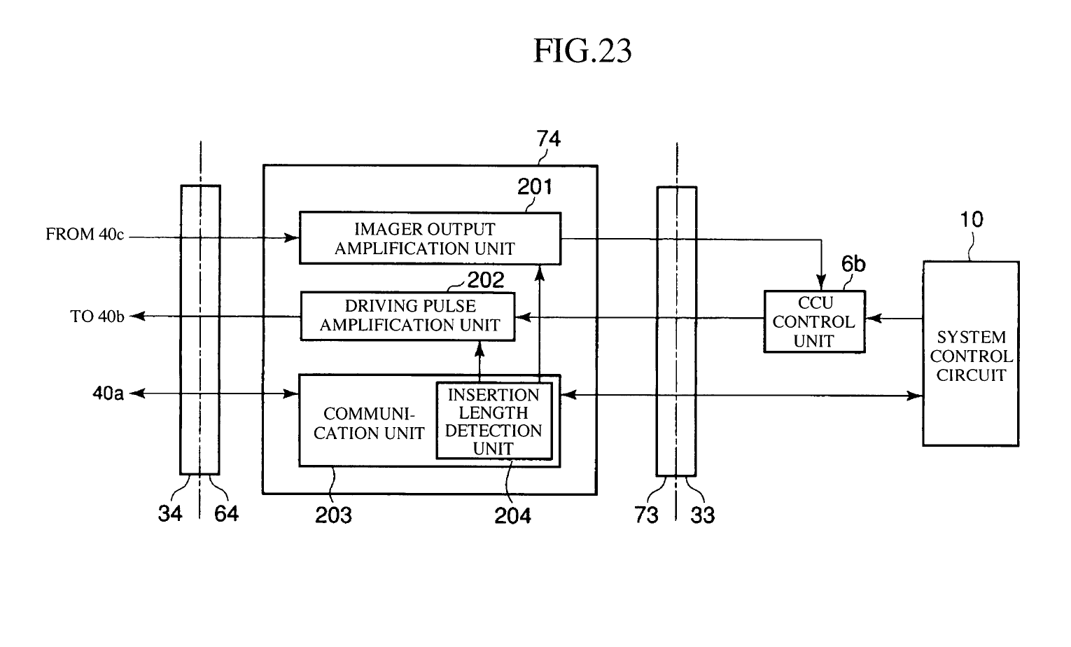

FIG. 23 is a block diagram showing the inner structure of the amplification circuit section 74. The amplification circuit section 74 comprises an imager output amplification unit 201, a driving pulse amplification unit 202, and a communication unit 203. Furthermore, the communication unit 203 comprises an insertion length detection unit 204.

The amplification circuit section 74 is connected respectively to the amplification side electrical connector 64 through a harness 81, and to the amplification side electrical connector 73 through another harness 82. Signals from the amplification side electrical connector 64 are to be input through the harness 81 to the amplification circuit section 74. Signals from the amplification side electrical connector 73 are to be input through the harness 82 to the amplification circuit section 74.

Moreover, when the second scope unit 2B, the amplification unit 60, and the fixed unit 3 are connected, the input side of the imager output amplification unit 201 is connected to the solid-state image sensing device 108 through the signal conductor 40c. Furthermore, the output side of the imager output amplification unit 201 is connected to the CCU control unit 6b. The input side of the driving pulse amplification unit 202 is connected to the CCU control unit 6b. Furthermore, the output side of the driving pulse amplification unit 202 is connected to the solid-state image sensing device 108 through the signal conductor 40b. Moreover, the communication unit 203 is connected to the electrical curve control unit 6a through the signal conductor 40a, and is connected to the system control circuit 10.

Furthermore, driving pulse signals from the CCU control unit 6b are input to the driving pulse amplification unit 202, and sent to the signal conductor 40b after a desired amplification processing. The imager outputs from the solid-state image sensing device 108 are relayed by the insertion section drive unit 5 and the electrical curve control unit 6a, and input to the imager output amplification unit 201 through the signal conductor 40c. The imager outputs which were input are sent to the CCU control unit 6b after the desired amplification processing, and converted into standard television signals by the CCU control unit 6b. The communication unit 203 communicates between the system control circuit 10 and the electrical curve control unit 6a.

The electrical curve signals which are the outputs of the system control circuit 10 are relayed by the communication unit 203 and sent to the electrical curve control unit 6a, and then converted into motor control signals. Various error values such as communication error, occurrence of motor overload, and the like which are detected by the electrical curve control unit 6a, are relayed by the communication unit 203 and sent to the system control circuit 10.

The connection detection conductor having the same structure as the abovementioned connection detection conductor between the fixed unit 3 and the scope unit 2, can detect whether or not between the fixed unit 3 and the amplification unit 60 are connected normally, and between the amplification unit 60 and the scope unit 2 are connected normally.

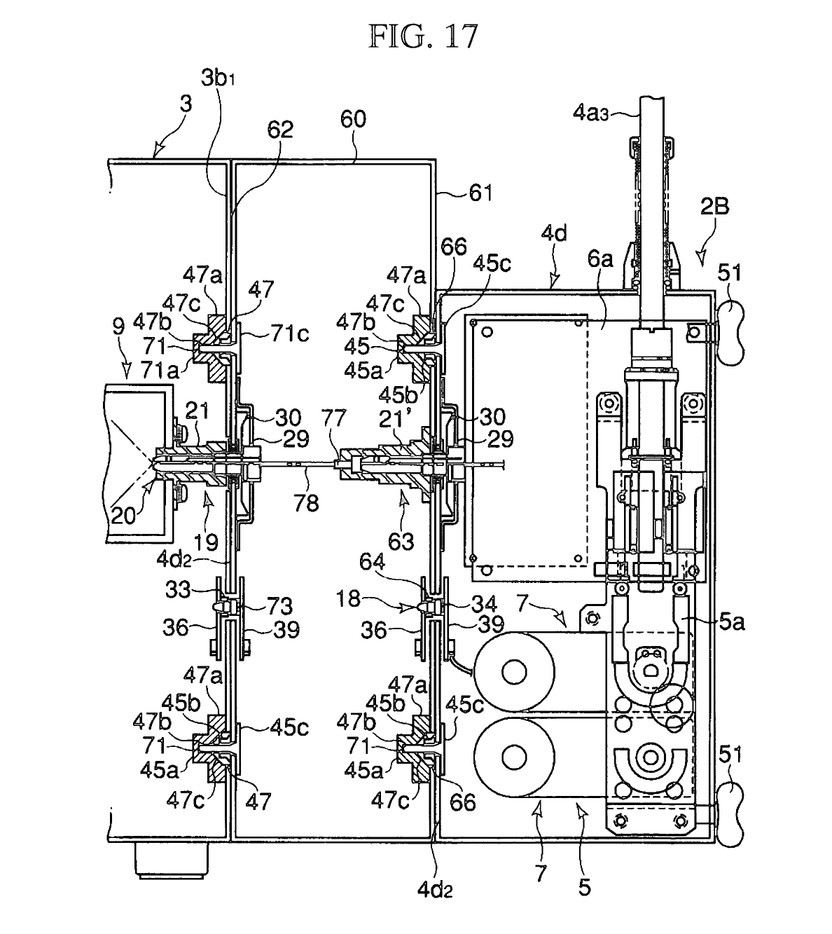

Also, as shown in FIG. 17, amplification side guide pin receivers 66 are attached in positions corresponding to the guide pins 45 of the base unit 4d on the base unit assembling surface 61 of the amplification unit 60 with good location accuracy. The amplification side guide pin receiver 66 has the same structure as that of the guide pin receiver 47 of the fixed unit 3. Therefore, the same reference symbols are used for components the same as those for the guide pin receiver 47 and description thereof is omitted here.

Furthermore, amplification side guide pins 71 are arranged in the position corresponding to the guide pin receivers 47 on the fixed unit 3 side on the fixed unit assembling surface 62. The amplification side guide pin 71 has the same structure as that of the guide pin 45. Therefore, the same reference symbols are used for components the same as those for the guide pin 45 and description thereof is omitted here.

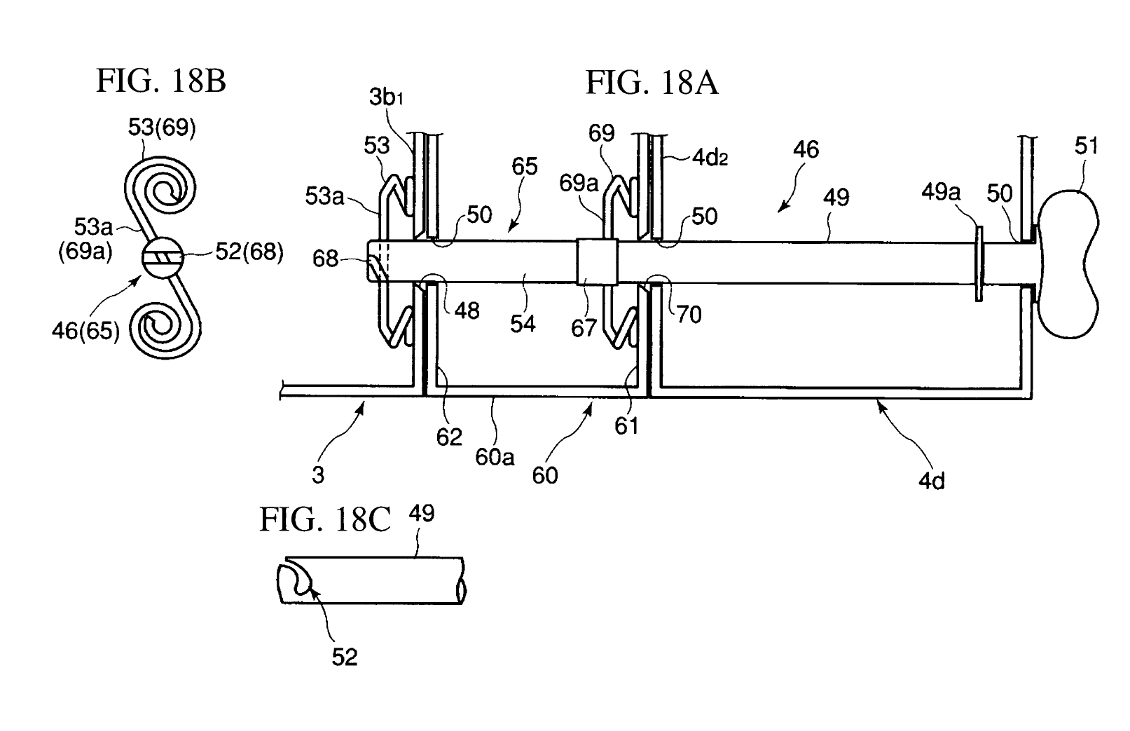

As shown in FIG. 18A, a lock coupler mechanism 65 which fastens and fixes when two box bodies of the fixed unit 3 and the base unit 4d of the second scope unit 2B are connected, is provided in the amplification unit 60. A connection shaft 54 which is connected through the tip end of the lock member 46 of the base unit 4d and an engaging section 67, is provided in the lock coupler mechanism 65. A locking groove 68 having a similar structure to that of the locking groove 52 of the shaft 49, is formed on the tip end of the connection shaft 54.

Furthermore, shaft insertion holes 70 are respectively formed in the base unit assembling surface 61 and the fixed unit assembling surface 62 of the unit case 60a of the amplification unit 60. A spring member 69 for locking the lock member 46 is fixed on the reverse face of the base unit assembling surface 61 around the shaft insertion hole 70 of the base unit assembling surface 61. The spring member 69 has a similar structure to that of the spring member 53 on the fixed unit 3 side. As shown in FIG. 18B, a straight locking section 69a is formed in the spring member 69. The locking groove 52 at the tip of the shaft 49 of the lock member 46 is to be unlockably locked to the locking section 69a. Moreover, similarly to the spring member 53 on the fixed unit 3 side, the locking groove 68 of the connection shaft 54 is to be unlockably locked.

Also, as shown in FIG. 4 and FIG. 9, two protruding resin slider members 76, each of which extends in the substantially horizontal direction, are vertically arranged on the side panel of the unit case 60a of the amplification unit 60; The slider members 76 have a similar structure to that of the slider members 13 of the base unit 4d.

For the harnesses 81 and 82 which exchange signals between the amplification circuit section 74, the amplification side electrical connector 64 and the amplification side electrical connector 73, the number of pins may be increased so as not to affect the case where the fixed unit 3 and the first scope unit 2A are assembled,. In this case, the arrangement may be such that the fixed unit side electrical connector 33 and the scope side electrical connector 34 of the first scope unit 2A having the increased number of pins are previously used and the pin used for the amplification unit 60 may be left unused.

Moreover, similarly to the first scope unit 2A, the scope having the short insertion length is suitable for shaping the drive wave forms which drive the solid-state image sensing device 108 such as the CCD in the structure of FIG. 1. However, similarly to the second scope unit 2B, using the scope having the long insertion length, the solid-state image sensing device 108 such as the CCD can not be sufficiently driven if the drive wave form is the same. Therefore, the amplification circuit section 74 comprises an insertion length detection unit 204 which detects the insertion length of the scope unit so that the amplification parameter can be variable according to the insertion length.

Next is a description of the operation of the above structure. The base unit 4d of the scope unit 2 is detachably connected to the junction 3b of the unit case 3a of the fixed unit 3 when the endoscope apparatus 1 of the present embodiment is used. Here, if desired to use the scope of the short insertion length type, the first scope unit 2A is attached.

First is a description of a case where the first scope unit 2A is attached. The slider members 13 of the base unit 4d of the first scope unit 2A are inserted into the guide rails 14 of the fixed unit 3 at the time of the connecting operation for the base unit 4d. In this state, the base unit 4d of the first scope unit 2A is made to slide along the guide rails 14 toward the fixed unit 3 side. Firstly, the LG connector 20 is contacted with the optical connector receiver 19 on the light source side 19 of the fixed unit 3 at the time of the sliding operation. At this time, the LG connector 20 slightly moves in the direction orthogonal to the axial direction (X-Y direction). Subsequently when the base unit 4d is pushed in in such a state, the LG connector 20 enters the connector block 21 of the optical connector receiver on the light source side 19.

When the tip end face of the LG connector 20 comes to the predetermined position where the light of the lamp in the light source unit 9 in the fixed unit 3 is focused, the step section 23b midway along the connector block 21 and the step section 27b midway along the LG connector 20 come up against each other.

When the LG connector 20 is pushed in further from this position, the LG connector 20 reinforced by the plate spring member 30 stays as it is and only the plate spring member 30 is elastically deformed in the depressed form. Therefore, when one fixed unit 3 and a plurality of models of scope units 2 are selectively combined, the position of the end face of the LG connector 20 is always maintained in the same position even if the plurality of scope units 2 are individually different.

Furthermore, during the connecting operation of the LG connector 20 and the connector block 21, the main body 47a of the guide pin receiver 47 and the shaft member of the guide pin 45 on the scope unit 2 side are contacted with each other, accompanied by the operation to push the base unit 4d of the scope unit 2A towards the fixed unit 3. At this time, the tapered surface 47c of the guide pin receiver 47 and the taper section 45a of the guide pin 45 come up against each other, so that the tip end of the guide pin 45 is smoothly inserted and fitted into the pin insertion hole 47b. Accordingly, the positional relationship between the axial direction of the fixed unit 3 and the base unit 4d of the scope unit 2A (Z direction) and the direction orthogonal to the axial direction (X-Y direction) is determined.

Subsequently, the fixed unit side electrical connector 33 and the scope side electrical connector 34 of the electrical connector unit 18 are connected. At the time of connecting the electrical connector unit 18, firstly the connector hollows 35a of the fixed unit side electrical connector 33 and the connector projections 38a of the scope side electrical connector 34 come up against each other.

At this time, the scope side electrical connector 34 slightly moves in the direction orthogonal to the axial direction (X-Y direction) due to the hollows and projections, and the connector projections 38a of the electrical connector main body 38 are inserted into the two connector hollows 35a of the fixed unit side electrical connector 33. In this state, when the scope unit 2A is further pushed in, the fixed unit side electrical connector 33 and the scope side electrical connector 34 fit with each other and the both contact points come in contact with each other and conduct.

The fixed unit side electrical connector 33 and the scope side electrical connector 34 which are integrally connected in this manner, will not move and reliable conduction is maintained even if vibration or the like is applied.

After this, the lock member 46 is used. When the lock member 46 is used, the base unit 4d of the scope unit 2 is abutted against the connection surface 3b1 of the fixed unit 3, and the knob 51 is rotated by hand while pushing the shaft 49.

At this time, the locking section 53a of the spring member 53 on the box body panel reverse face of the connection surface 3b1 is fitted into the locking groove 52 at the tip of the shaft 49. In this state, if the shaft 49 is further rotated, the locking section 53a of the spring member 53 is pulled in by the locking groove 52 of the shaft 49 so that the locking section 53 is reliably locked at the end of the locking groove 52. At this time, since the engaging section of the locking groove 52 of the shaft 49 and the locking section 53a of the spring member 53 is always reinforced by the spring force of the spring member 53, the lock member 46 will not be unlocked until the shaft 49 is reversibly rotated so as to unlock it. Then, in this state, the illumination light from the light source unit 9 is transmitted from the optical connector 17 for optical connections via the light guide 28, and irradiates the front subject by the illumination optical system 104 placed in the tip end head section 4a1 of the first scope unit 2A. Accordingly, the examination is performed using the endoscope apparatus 1.

Moreover, in the case where the examination can not be achieved by the first scope unit 2A, for example if desired to observe inside a long pipe such as in a plant, the first scope unit 2A must be replaced by the second scope unit 2B having the long insertion length. In this case, as shown in FIG. 2, the base unit 4d of the second scope unit 2B is assembled to the fixed unit 3 through the amplification unit 60.

Firstly, the connecting operation of the amplification unit 60 to the fixed unit 3 is performed at the time of the connecting operation for the base unit 4d of the second scope unit 2B. The slider members 76 of the amplification unit 60 are inserted into the guide rails 14 of the fixed unit 3 at the time of the connecting operation of the amplification unit 60. In this state, the amplification unit 60 is made to slide along the guide rails 14 toward the fixed unit 3 side. Firstly, the amplification side optical connector 72 is contacted with the connector block 21 of the fixed unit 3 at the time of the sliding operation.

At this time, the amplification side optical connector 72 slightly moves in the direction orthogonal to the axial direction (X-Y direction). When the amplification unit 60 is further pushed in in such a state, the amplification side optical connector 72 enters the connector block 21.

When the tip end face of the amplification side optical connector 72 comes to the predetermined position where the light of the lamp in the light source unit 9 in the fixed unit 3 is focused, the step section 23b midway along the connector block 21 and the step section 27b midway along the amplification side optical connector 72 come up against each other.

When the amplification side optical connector 72 is pushed in further from this position, the amplification side optical connector 72 reinforced by the plate spring member 30 stays as it is and only the plate spring member 30 is elastically deformed in the depressed form. Therefore, when one fixed unit 3 and a plurality of models of scope units 2 are selectively combined, the position of the end face of the amplification side optical connector 72 is always maintained in the same position even if the plurality of scope units 2 are individually different.

Furthermore, during the connecting operation of the amplification side optical connector 72 and the connector block 21, the main body 47a of the guide pin receiver 47 and the shaft member of the amplification side guide pin 71 on the amplification unit 60 side are contacted with each other, accompanied by the operation to push the amplification unit 60 towards the fixed unit 3. At this time, the tapered surface 47c of the guide pin receiver 47 and the taper section 45a of the amplification side guide pin 71 come up against each other, so that the tip end of the amplification side guide pin 71 is smoothly inserted and installed into the pin insertion hole 47b. Accordingly, the positional relationship between the axial direction of the fixed unit 3 and the amplification unit 60 (Z direction) and the direction orthogonal to the axial direction (X-Y direction) is determined.

Subsequently, the fixed unit side electrical connector 33 and the male type amplification side electrical connector 73 are connected. At the time of connecting the electrical connectors, firstly the connector hollows 35a of the fixed unit side electrical connector 33 and the connector projections 38a of the amplification side electrical connector 73 come up against each other.

At this time, the amplification side electrical connector 73 slightly moves in the direction orthogonal to the axial direction (X-Y direction) due to the hollows and projections, and the connector projections 38a of the electrical connector main body 38 are inserted into the two connector hollows 35a of the electrical connector on the fixed unit side main body 38. In this state, when the amplification unit 60 is further pushed in, the fixed unit side electrical connector 33 and the amplification side electrical connector 73 fit with each other and the both contact points come in contact with each other and conduct. The fixed unit side electrical connector 33 and the amplification side electrical connector 73 which are integrally connected in this manner, will not move and reliable conduction is maintained even if vibration or the like is applied.

After finishing the connecting operation of the amplification unit 60 to the fixed unit 3, next, the attaching operation of the base unit 4d of the second scope unit 2B to the amplification unit 60 is performed. The slider members 13 of the scope unit 2B are inserted into the guide rails 14 of the fixed unit 3 at the time of the connecting operation of the base unit 4d. In this state, the scope unit 2B is made slide to along the guide rails 14 toward the amplification unit 60 side. Firstly, the LG connector 20 of the scope unit 2B is contacted with the connector block 21′ of the amplification unit 60 at the time of the sliding operation.

At this time, the LG connector 20 slightly moves in the direction orthogonal to the axial direction (X-Y direction). When the scope unit 2B is further pushed in in such a state, the LG connector 20 enters the connector block 21.

During the inserting operation of the LG connector 20, the step section 23b′ midway along the connector block 21′ and the step section 27b midway along the LG connector 20 come up against each other.

When the LG connector 20 is pushed in further from this position, the LG connector 20 reinforced by the plate spring member 30 stays as it is and only the plate spring member 30 is elastically deformed in the depressed form. Therefore, when one amplification unit 60 and a plurality of models of scope units 2 are selectively combined, the position of the end face of the LG connector 20 is always maintained in the same position even if the plurality of scope units 2 are individually different.

Furthermore, during the connecting operation of the LG connector 20 and the connector block 21′, the main body 47a of the amplification side guide pin receiver 66 and the shaft member of the guide pin 45 on the scope unit 2B side are contacted with each other, accompanied by the operation to push the base unit 4d of the scope unit 2B towards the amplification unit 60. At this time, the tapered surface 47c of the amplification side guide pin receiver 66 and the taper section 45a of the guide pin 45 come up against each other, so that the tip end of the guide pin 45 is smoothly inserted and fitted into the pin insertion hole 47b. Accordingly, the positional relationship between the axial direction of the amplification unit 60 and the base unit 4d of the scope unit 2B (Z direction) and the direction orthogonal to the axial direction (X-Y direction) is determined.

Subsequently, the female type electrical connector on the amplification unit side 64 and the scope side electrical connector 34 are connected. At the time of the connection, firstly the connector hollows 35a of the female type electrical connector on the amplification unit side 64 and the connector projections 38a of the scope side electrical connector 34 come up against each other.

At this time, the scope side electrical connector 34 slightly moves in the direction orthogonal to the axial direction (X-Y direction) due to the hollows and projections, and the connector projections 38a of the electrical connector main body 38 are inserted into the two connector hollows 35a of the female type electrical connector on the amplification unit side 64. In this state, when the scope unit 2B is further pushed in, the female type electrical connector on the amplification unit side 64 and the scope side electrical connector 34 fit with each other and the both contact points come in contact with each other and conduct.

The female type electrical connector on the amplification unit side 64 and the scope side electrical connector 34 which are integrally connected in this manner, will not move and reliable conduction is maintained even if vibration or the like is applied.

After this, the lock member 46 is used. When the lock member 46 is used, the base unit 4d of the scope unit 2 comes up against the connection surface 3b1 of the fixed unit 3 via the amplification unit 60, and the knob 51 is rotated by hand while pushing the shaft 49.

At this time, the locking groove 52 at the tip of the shaft 49, and the engaging section 67 of the lock coupler mechanism 65 are engaged, and the locking section 53a of the spring member 53 on the box body panel reverse face of the connection surface 3b1 is fitted into the locking groove 68 at the tip of the connection shaft 54.

In this state, if the shaft 49 is further rotated, the locking section 53a of the spring member 53 is pulled in by the locking groove 68 so that the locking section 53 is reliably locked at the end of the locking groove 68. At this time, since the engaging section of the locking groove 68 of the connection shaft 54 and the locking section 53a of the spring member 53 is always reinforced by the spring force of the spring member 53, the lock member 46 will not be unlocked until the shaft 49 is reversibly rotated so as to unlock the locking groove 68 and release the engagement of the engaging section 67.

The endoscope apparatus 1 is driven in this state. At this time, the illumination light from the light source unit 9 is guided from the joint of the light source side optical connector 19 and the amplification side optical connector 72 via the relay LG bundle 78 to the side of the amplification side optical connector 63. Consequently, the illumination light is transmitted from the optical connector 17 for optical connections between the amplification side optical connector 63 and the LG connector 20 via the light guide 28. Then, the illumination light irradiates the front subject by the illumination optical system 104 placed in the tip end head section 4a1 of the second scope unit 2B. Accordingly, the examination is performed using the endoscope apparatus 1.

Moreover, when the second scope unit 2B, the amplification unit 60, and the fixed unit 3 are connected, the amplification circuit section 74 operates in the following manner. The driving pulse signals from the CCU control unit 6b are input to the driving pulse amplification unit 202 and sent to the signal conductor 40b after the desired amplification processing.

The imager outputs from the solid-state image sensing device 108 are relayed by the insertion section drive unit 5 and the electrical curve control unit 6a, and input to the imager output amplification unit 201 through the signal conductor 40c. The imager outputs which were input are sent to the CCU control unit 6b after the desired amplification processing, and converted into standard television signals by the CCU control unit 6b. The communication unit 203 communicates between the system control circuit 10 and the electrical curve control unit 6a. The electrical curve signals which are the outputs of the system control circuit 10 are relayed by the communication unit 203 and sent to the electrical curve control unit 6a, and then converted into motor control signals.

Various error values such as communication error, occurrence of motor overload, and the like which are detected by the electrical curve control unit 6a, are relayed by the communication unit 203 and sent to the system control circuit 10.

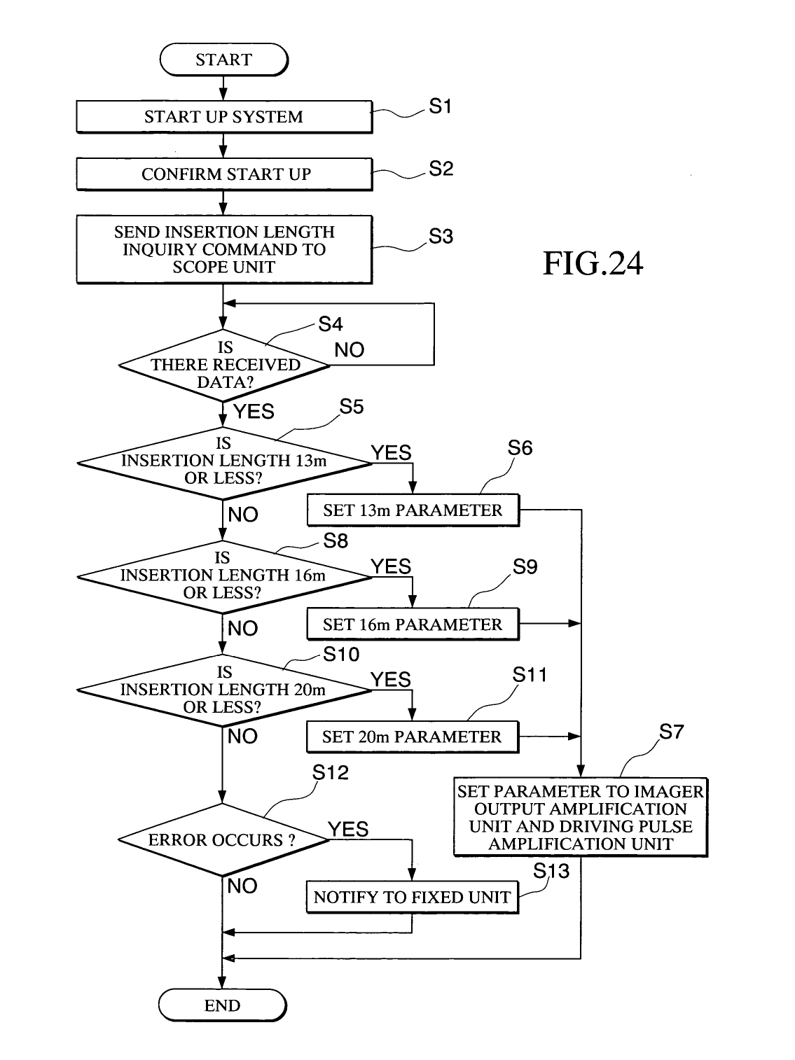

Moreover, when starting up the endoscope apparatus 1, the amplification unit 60 sends an insertion length inquiry command with respect to the scope unit 2B. The operation of the amplification unit 60 at this time is described with reference to the flow chart in FIG. 24.

Firstly, the system is started up by pressing the POWER button of the remote control (step S1). At this time, the amplification unit 60 also performs communication for confirming start-up between the system control circuit 10 and the electrical curve control unit 6a.

In the next step S2, when the start-up of the system control circuit 10 and the scope unit 2B is confirmed, the insertion length inquiry command with respect to the scope unit 2B is sent (step S3), and the response is awaited in the reception waiting loop.

When the received data is received (step S4), the insertion length included in the received data is confirmed (step S5). If in step S5 the insertion length is 13 m or less, the flow proceeds to step S6 and the 13 m parameter is set. Subsequently, the flow proceeds to step S7 and the above parameter is set to the imager output amplification unit 201 and the driving pulse amplification unit 202.

In step S5, if the insertion length is not 13 m or less, the flow proceeds to step S8. In the step S8, if the insertion length is 16 m or less, the flow proceeds to step S9 and the 16 m parameter is set. Subsequently, the flow proceeds to step S7 and the above parameter is set in the imager output amplification unit 201 and the driving pulse amplification unit 202.

In step S8, if the insertion length is not 16 m or less, the flow proceeds to step S10. In the step S10, if the insertion length is 20 m or less, the flow proceeds to step S11 and the 20 m parameter is set. Subsequently, the flow proceeds to step S7 and the above parameter is set in the imager output amplification unit 201 and the driving pulse amplification unit 202.

The imager outputs and the driving pulses are respectively amplified in the imager output amplification unit 201 and the driving pulse amplification unit 202 based on the above parameter.

In step S10, if the insertion length is not 20 m or less, the flow proceeds to step S12. In step S12, the existence/nonexistence of error occurrences is judged. In step S12, if the received data does not include the insertion length but includes error data, the flow proceeds to step S13 and this is notified to the fixed unit 3.

In the fixed unit 3, the system control circuit 10 receives and displays the desired error processing, for example, the error data, on the monitor screen, and beeps.

When the scope unit 2B is connected, the CCD drive wave forms of the CCU control unit 6b are amplified by the amplification circuit section 74, and transmitted to the solid-state image sensing device 108 of the CCD via the amplification side electrical connector 64 and the scope side connector 34.

Moreover, similarly, the electric signals which are transmitted from the solid-state image sensing device 108 of the CCD (converted into picture signals thereafter) are also amplified so as to eliminate signal deterioration due to the long insertion section 4a, and sent to the CCU control unit 6b.

Here, the following effects are demonstrated in the above structure. That is, the CCU control unit 6b is loaded on the fixed unit side in the system of the endoscope apparatus 1 of the present embodiment. Therefore, it contributes to lighten and reduce the cost of the scope unit 2 by not providing the CCU control unit 6b on the scope unit 2 side. Furthermore, in the case where the long scope unit 2B which can not be corresponded with the CCU control unit 6b on the fixed unit 3 side is used, by connecting to the fixed unit 3 through the amplification unit 60, a system which can correspond to various models can be provided.

That is, in the case where the amplification circuit sections 74 are respectively provided in the base units 4d of the scope units 2B, the amplification circuit sections 74 should be loaded to all of the scope units 2B which can not be corresponded with the CCU control unit 6b. However, the scope units 2B which can not corresponded with the CCU control unit 6b can be usable by providing one amplification circuit section 74 for the amplification unit 60.

Specifically, preferably the amplification unit 60 is not used for the scope unit 2A having the insertion length less than 10 m, and the scope units having the insertion length of 10 m or more, for example such as the scope unit 2B of 13 m, 16 m and 20 m, are connected through the amplification unit 60.

That is, the three types of scope unit 2B of 13 m, 16 m and 20 m, become usable by having only one amplification unit 60. Therefore, many functions of the endoscope can be widened, strengthened, and added, the usable functions can be expanded, labor for the examination preparation and the examination itself can be saved, and the cost of the whole system can be reduced.

(Second Embodiment)

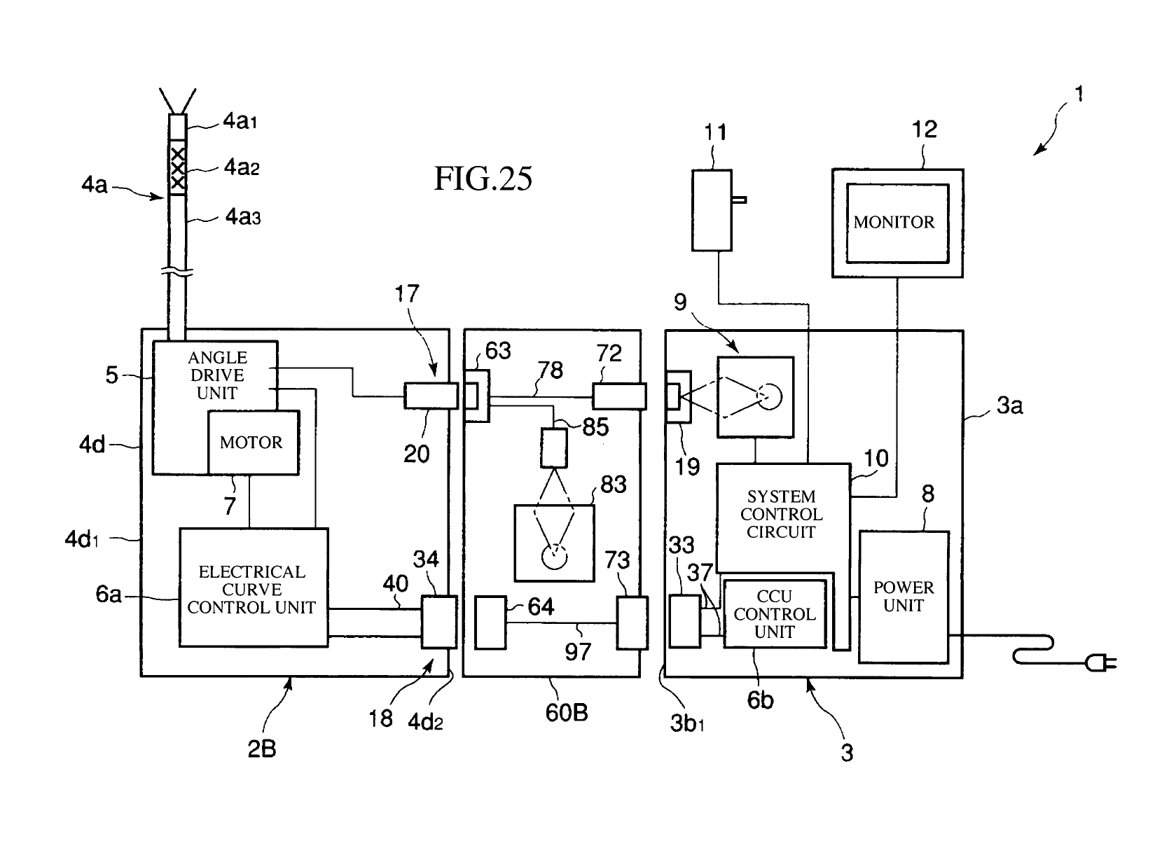

FIG. 25 to FIGS. 27A and B show a second embodiment of the present invention. The present embodiment is provided with an amplification unit 60B having a different structure from that of the amplification unit 60 of the endoscope apparatus 1 of the first embodiment (refer to FIG. 1 to FIG. 24). The structure of the other parts is the same as that of the endoscope apparatus 1 of the first embodiment. Therefore, the same reference symbols are used for components the same as those for the endoscope apparatus 1 of the first embodiment and description thereof is omitted here.

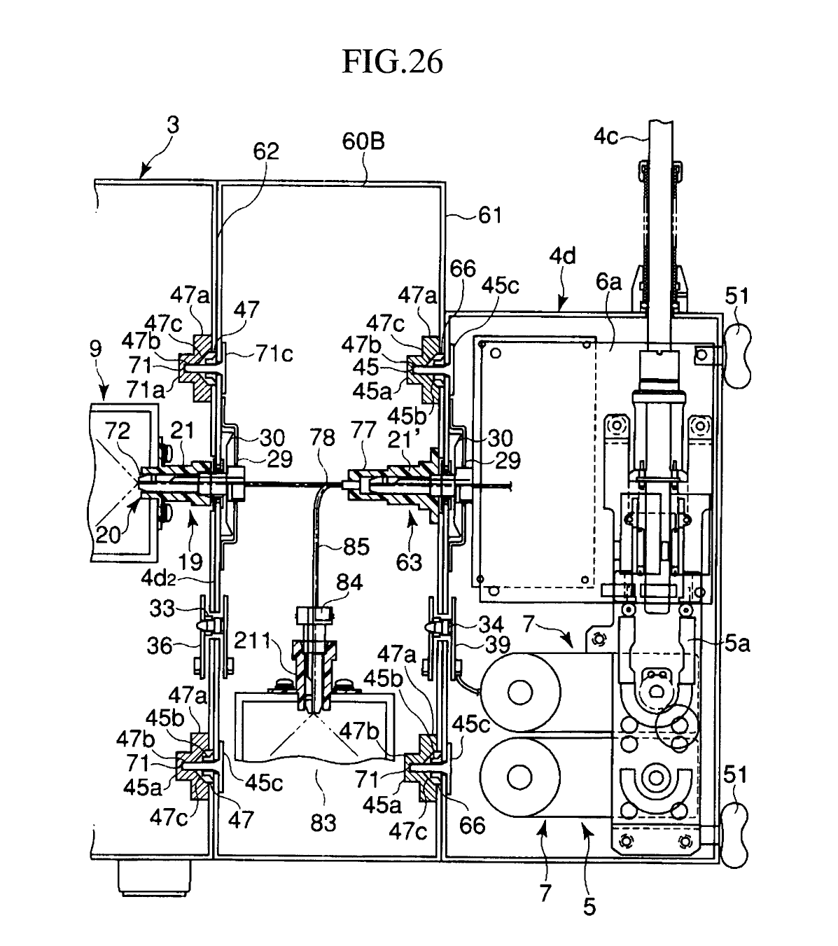

That is, as shown in FIG. 25, a second light source unit 83 is provided in the amplification unit 60B in the present embodiment. As shown in FIG. 26, a light source side optical connector 211 having a similar structure to that of the light source side optical connector 19 is provided in the light source unit 83.

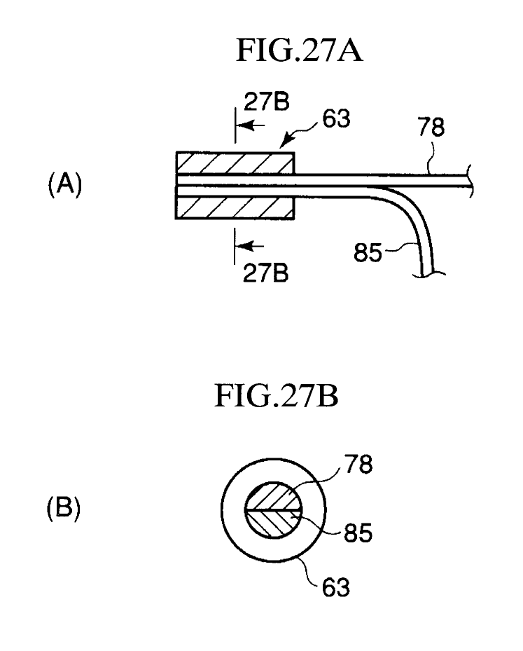

Moreover, as shown in FIG. 27A and B, one end of the relay LG bundle 78 and one end of the second LG bundle 85 are connected together to the amplification side optical connector 63. The LG connector 84 is connected to the other end of the second LG bundle 85. The LG connector 84 is connected to the light source side optical connector 211 of the second light source unit 83. Furthermore, the illumination light of the second light source unit 83 can be transmitted to the amplification side optical connector 63 through the second LG bundle 85.

Next is a description of the operation of the present embodiment of the above structure. In the present embodiment, as shown in FIG. 26, when the base unit 4d of the second scope unit 2B is assembled with the fixed unit 3 through the amplification unit 60B, a second illumination light which is transmitted from the second light source unit 83 through the LG connector 84 and the LG bundle 85, is transmitted to the amplification side optical connector 63, in a state of merging into the illumination light which is transmitted through the relay LG bundle 78. Accordingly, illumination light which is the sum of the normal illumination light from the light source unit 9 and the second illumination light from the second light source unit 83, and thus has an increased light quantity, irradiates the specimen from the tip end of the second scope unit 2B.

Here, in the present embodiment, the illuminating function is increased by the second light source unit 83 of the amplification unit 60B so that bright examination image can be obtained. As a result, there is an effect of decreasing eye fatigue of the examiner and increasing examination efficiency.

Moreover, the loss of the light quantity is large if the insertion length is long and the light guide in the scope is long. Therefore, in the case where the amplification unit 60B of the present function is loaded with respect to the scope unit 2B having such a long light guide, the lost light quantity can be compensated for, so that an image which does not impede the examination can be obtained.

(Third Embodiment)

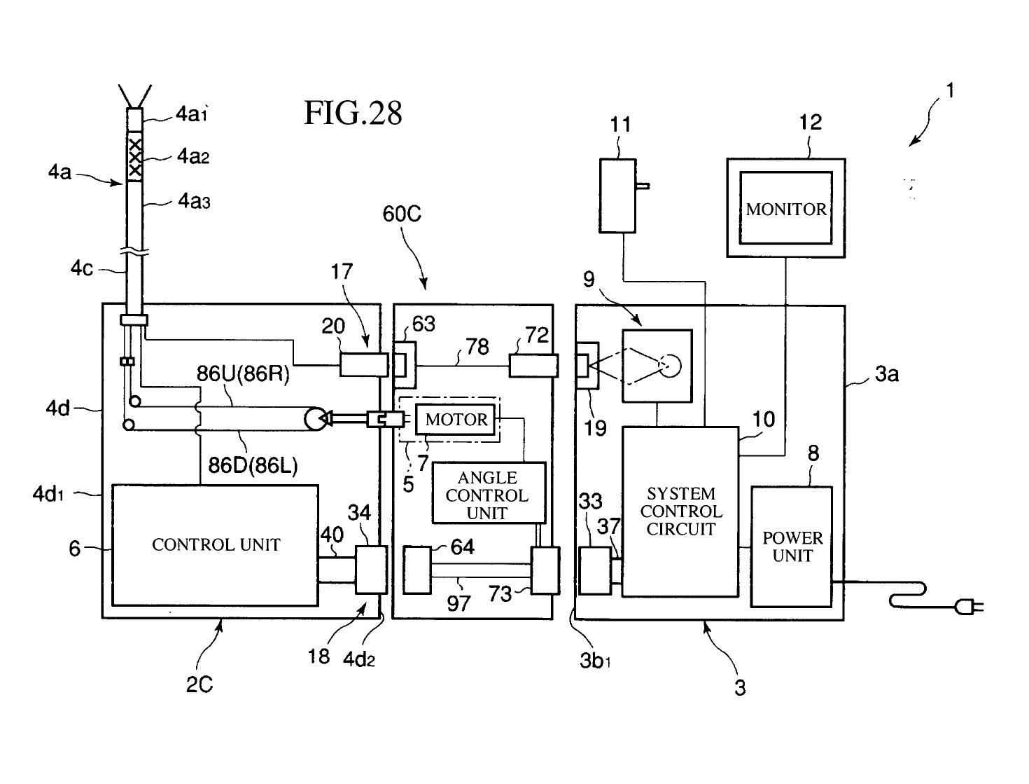

FIG. 28 to FIG. 30 show a third embodiment of the present invention. In the industrial endoscope, in some cases examination by converting the line of sight specifically by curving is not required for a long and thin subject of examination such as the piping of a chemical plant. In this case, rather than the type of the scope with the curving section which actively operates by angle wires or the like, one which presses the tip end to the outer wall of the pipe and passively curves the curving section by the external force is used.

In the present embodiment, there is a description of a case where a third scope unit 2C having a curving section 4a2 in the insertion section 4a, but not with the function to curve the curving section 4a2, is connected.

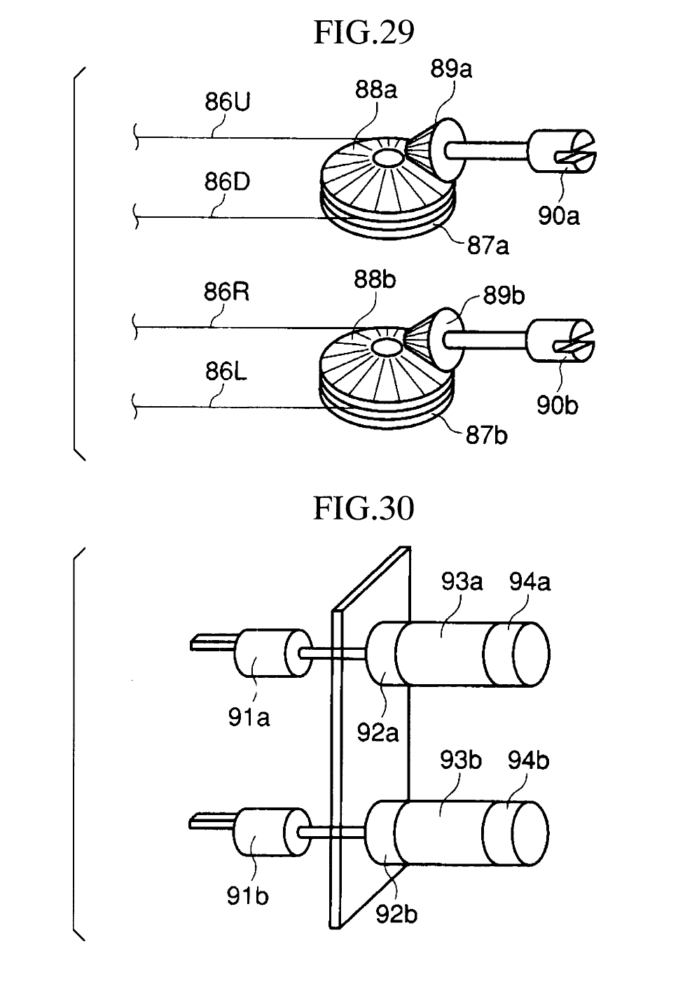

The insertion section 4a is provided for the third scope unit 2C similarly to the first scope unit 2A. The insertion section 4a has a head section 4a1, the curving section 4a2, and a flexible tube section 4a3. Moreover, a base unit 4d is provided at the base end of the insertion section 4a. As shown in FIG. 29, four angle wires 86 (angle wires 86U, 86D, 86R, and 86L) are arranged in the base unit 4d. Here, the angle wire 86U and the angle wire 86D are wound around one pulley section 87a and fixed. A bevel gear section 88a is provided on the top surface of the pulley section 87a. A driving bevel gear section 89a meshing with the bevel gear section 88a is provided as a set. A female type joint section 90 combined with the electrical curve drive unit 5 which will be described later is provided at the end of the bevel gear section 89a.

Furthermore, similarly, a pulley section 87b, a bevel gear section 88b, a driving bevel gear section 89b, and a female type joint section 90b are respectively provided for the angle wire 86R and the angle wire 86L.

Next is a description of an amplification unit 60C of the present embodiment. In the amplification unit 60C is provided an electrical curve drive unit 5 having male type joint sections 91a and 91b as shown in FIG. 30, which are joined to the female type joint sections 90a and 90b.

A motor unit 7 is connected to each of the male type joint sections 91a and 91b. The motor unit 7 has deceleration gear sections 92, motor sections 93, and encoder sections 94.

Next is a description of the operation of the present embodiment of the above structure. If the third scope unit 2C is not curved, the base unit 4d is directly docked to the fixed unit 3 as is. In this case, the amplification unit 60C is not used.

However, the insertion section 4a of the third scope unit 2C has the curving section 4b. Therefore, if an external force is applied to the head section 4a1, the insertion section 4a itself can be passably curved by the external force so that the line of sight can be converted. Therefore, although the insertion section 4a does not have the active curving function, it can be curved.

Furthermore, as shown in FIG. 28, regarding the curving section 4b of the insertion section 4a of the third scope unit 2C, in the case where the curving function is used in the active form, the base unit 4d is docked to the fixed unit 3 through the amplification unit 60C.

At this time, if the base unit 4d is docked to the amplification unit 60C, the female type joint sections 90a and 90b of the third scope unit 2C are joined to the male type joint sections 91a and 91b of the amplification unit 60.

Then, the rotation of the motor unit 7 is connected and transmitted by the female type joint sections 90 and 91, the rotation direction is converted by the bevel gear sections 88 and 89, and the angle wires 86 are tractionally operated.

Here, the following effects are demonstrated in the above structure. That is, if curving is not used in the present embodiment, the amplification unit 60C is not used. Therefore, since there is no motor unit 7 and the like in the base unit 4d of the third scope unit 2C in this case, this contributes to lightening and improvement in the portability of the system.

Furthermore, by using the amplification unit 60C, the curving section 4b of the third scope unit 2C can be actively used while amplifying the curving function by the electrical curve drive unit 5 in the amplification unit 60C. Accordingly, the system can be used in different ways according to the purpose.

(Modified Example of Third Embodiment)

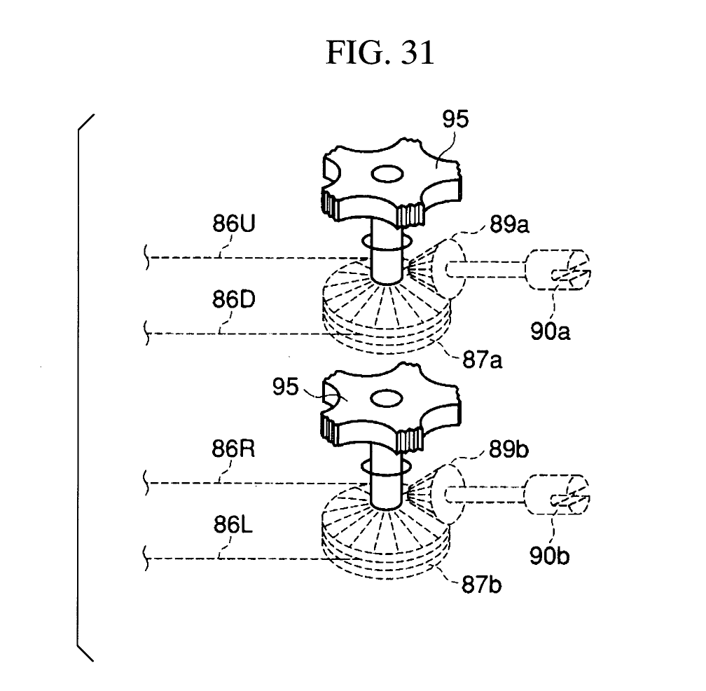

FIG. 31 shows a modified example of a pair of curve operation mechanisms in the base unit 4d of the third scope unit 2C of the third embodiment. In the present modified example, curved knobs 95 which are directly connected to each of pulley sections 87a and 87b in the base unit 4d of the third scope unit 2C, are provided so as to provide an active curving function in the third scope unit 2C.

In this case, if the amplification unit 60C is not used and the base unit 4d of the third scope unit 2C and the fixed unit 3 are directly docked, the system becomes one of manual curve operation. Moreover, if the amplification unit 60C is added and the base unit 4d of the third scope unit 2C and the fixed unit 3 are directly docked through the amplification unit 60C, the system becomes one of electrical curve operation. Therefore, in this case, the curving function may be amplified for the purpose of labor saving and improving the operational feeling.

The structure may be such that a manual curving device having the projecting knobs or the like is provided on the amplification unit 60C side, instead of the electrical curve drive unit 5.

(Fourth Embodiment)

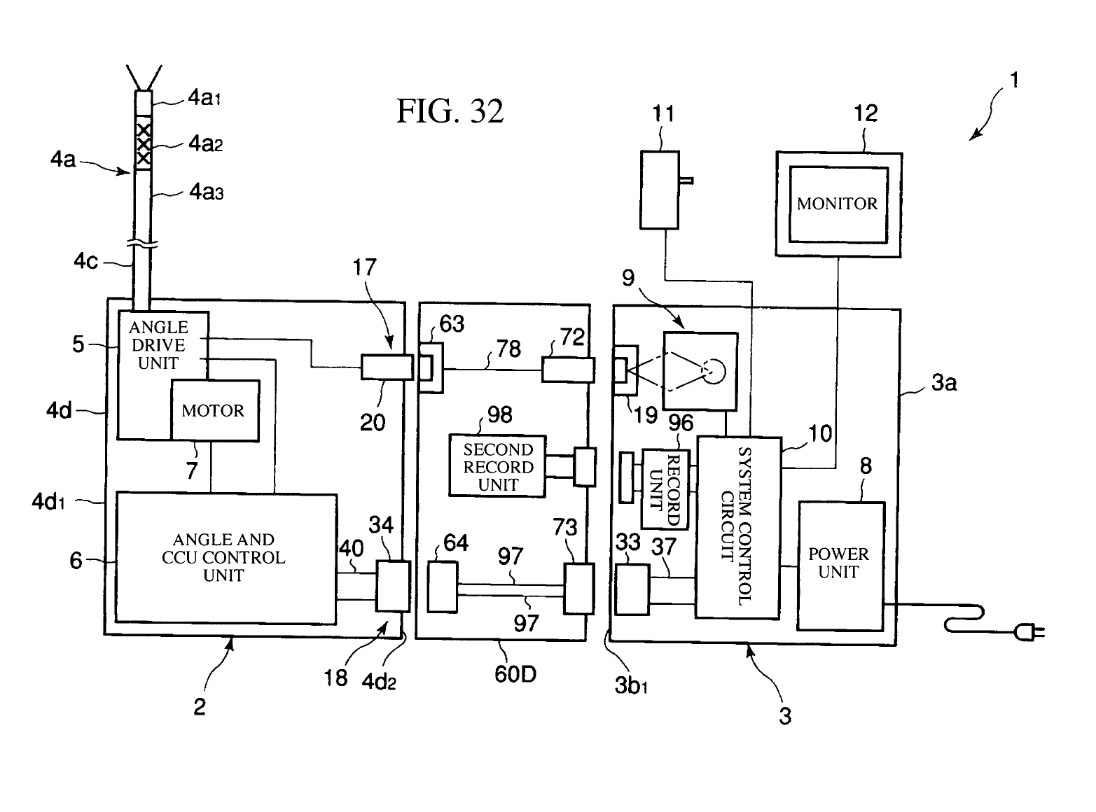

FIG. 32 shows a fourth embodiment of the present invention. This embodiment is provided with an amplification unit 60D having a different structure from that of the amplification unit 60 of the endoscope apparatus 1 of the first embodiment (refer to FIG. 1 to FIG. 24). The structure of the other parts is the same as that of the endoscope apparatus 1 of the first embodiment. Therefore, the same reference symbols are used for components the same as those for the endoscope apparatus 1 of the first embodiment and description thereof is omitted here.

That is, as shown in FIG. 32, in this embodiment, a second record unit 98 is provided in the amplification unit 60D, in addition to the record unit 96 which is the recording device built into the fixed unit 3 in the standard manner.

Next is a description of the operation of the above structure. In this embodiment, images and sound recordings which could not be stored by the record unit 96 of the fixed unit 3 can be stored by the second record unit 98.

Here, since the data can be respectively stored by the record unit 96 of the fixed unit 3 and the second record unit 98 in the above structure, the memory capacity can be increased. Accordingly, there is the effect of being able to store larger images, and being able to correspond to longer examinations, compared to with the conventional device.

(Fifth Embodiment)

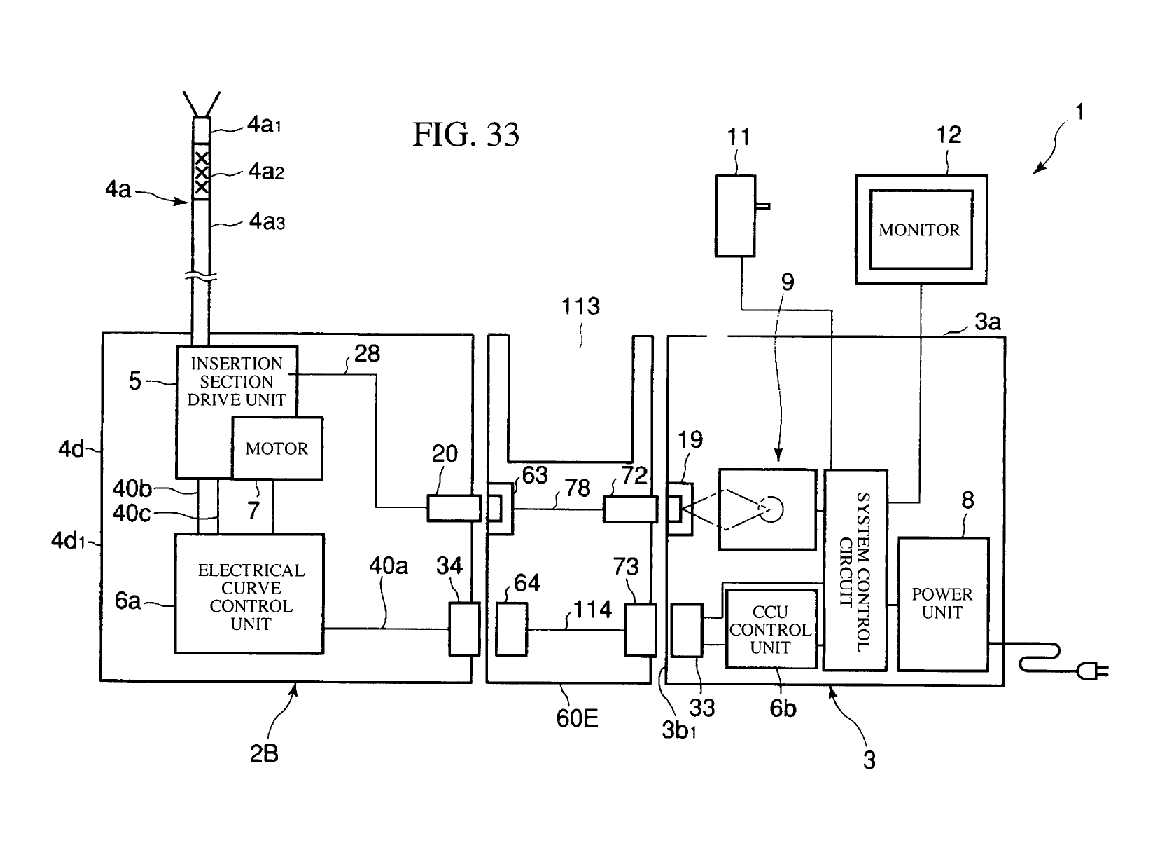

FIG. 33 to FIG. 35 show a fifth embodiment of the present invention. This embodiment is provided with an amplification unit 60E having a different structure from that of the amplification unit 60 of the endoscope apparatus 1 of the first embodiment (refer to FIG. 1 to FIG. 24). The structure of the other parts is the same as that of the endoscope apparatus 1 of the first embodiment. Therefore, the same reference symbols are used for components the same as those for the endoscope apparatus 1 of the first embodiment and description thereof is omitted here.

That is, as shown in FIG. 33, the amplification unit 60E of this embodiment has an amplification side optical connector 63, an amplification side optical connector 72, a relay LG bundle 78, a female type amplification side electrical connector 64, a male type amplification side electrical connector 73a and a harness 114. The amplification unit 60E further has an insertion section storage section 113 which stores an insertion section 4a of a scope unit 2.

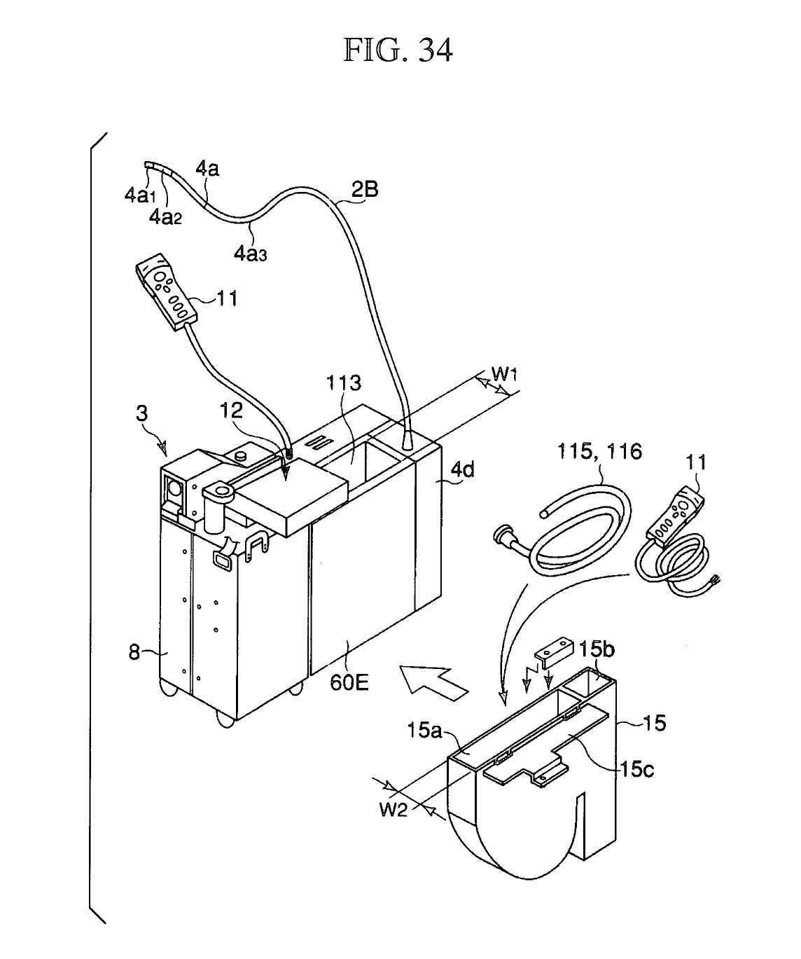

As shown in FIG. 34, the width W1 of the insertion section storage section 113 is broader than the width W2 of the scope storage section 15a of the storage section box 15 which is normally used.

Next is a description of the operation of this embodiment of the above structure. The amplification unit 60E and the base unit 4d are docked with the fixed unit 3.

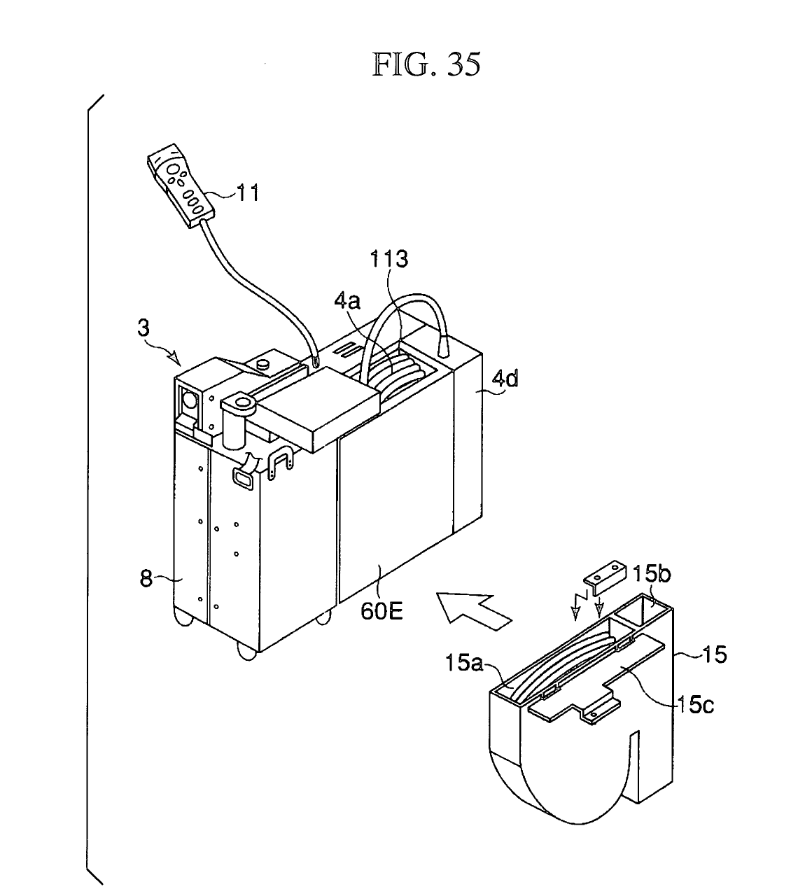

Then, as shown in FIG. 35, if the scope unit 2 is not used, the insertion section 4a of the scope unit 2 is stored in the insertion section storage section 113 of the amplification unit 60E.

In this embodiment, the scope unit 2B of the long insertion section 4a type is attached. Moreover, by having the insertion section storage section 113 of the width W1 which is broader than the width W2 of the scope storage section 15a of the storage section box 15 which is normally used, the wound long insertion section 4a is also stored in the broader insertion section storage section 113.

Here, the following effects are demonstrated in the above structure. That is, in this embodiment, the amplification unit 60E having the insertion section storage section 113 which increases the storing function, is provided so that even a scope having the insertion section 4a of the insertion length of 10 m or more may be easily stored. If the storage section is small, since the stored subject is long braid-shaped, there may be a case where the insertion section 4a is tangled unless it is carefully wound and stored in the storage section. On the other hand, since the insertion section storage section 113 is large, there is the effect of being able to omit operations which require excessive care. Moreover, a chemical resistant tube 115 which covers the insertion section 4a for the purpose of protecting the endoscope when inserting into a tank or pipe containing medicines or the like, a guide tube 116 which can freely change its shape, and guide the insertion section 4a to a desired position by inserting the insertion section 4a thereinside, or a spare remote control, may be stored in the storage section box 15 which is normally used.

(Sixth Embodiment)

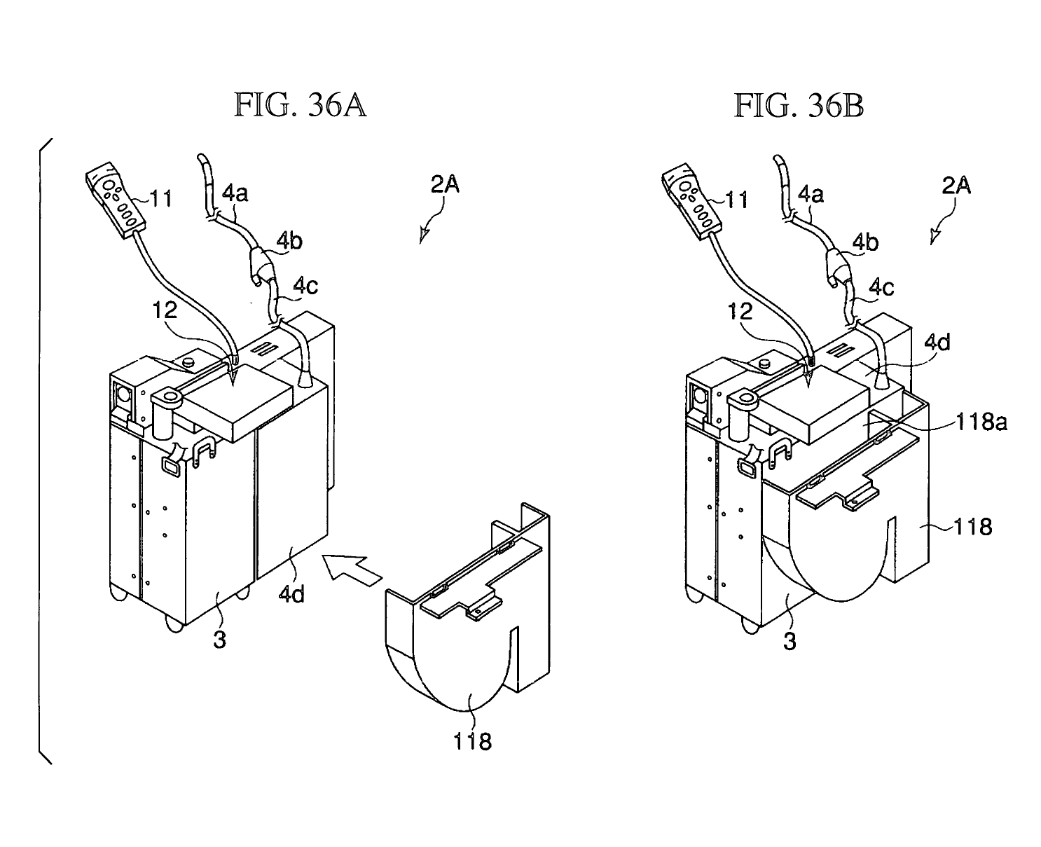

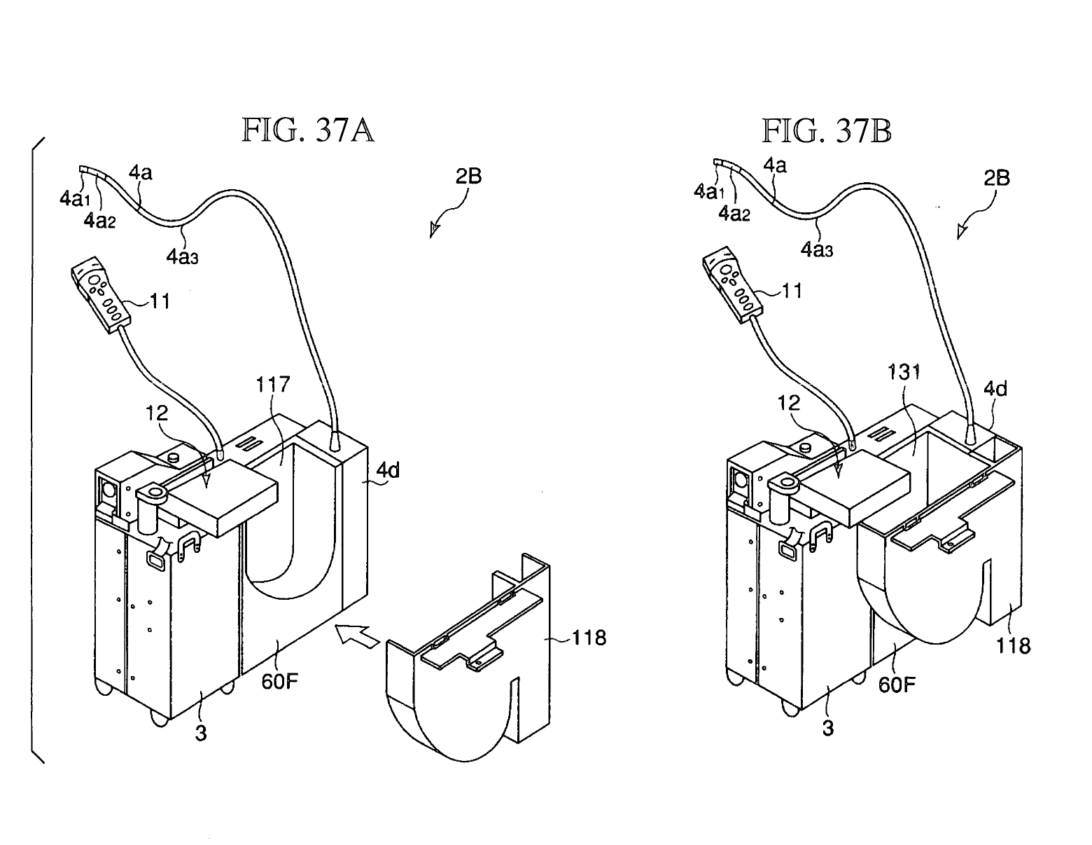

FIGS. 36A and B and FIGS. 37A and B show a sixth embodiment of the present invention. In this embodiment, the storage section box 15 of the endoscope apparatus 1 of the first embodiment (refer to FIG. 1 to FIG. 24) is modified into a storage box 118 which is notched on one face, and an amplification unit 60F having a different structure from that of the amplification unit 60 of the endoscope apparatus 1 of the first embodiment (refer to FIG. 1 to FIG. 24) is provided. The structure of the other parts is the same as that of the endoscope apparatus 1 of the first embodiment. Therefore, the same reference symbols are used for components the same as those for the endoscope apparatus 1 of the first embodiment and description thereof is omitted here.

That is, as shown in FIG. 37A, the amplification unit 60F of the present embodiment has an indented shape storage hollow 117 having one side opened. As shown in FIGS. 36A and B, when the first scope unit 2A is assembled to the fixed unit 3, the amplification unit 60F is not used. In this case, as shown in FIG. 36A, by using the storage box 118 which is notched on one face, in combination with the wall face of the fixed unit 3 and the base unit 4d of the first scope unit 2A, as shown in FIG. 36B a box-shaped storage space 118a is formed. Normally, the insertion section 4a, the intermediate connection section 4b and the universal cable 4c of the first scope unit 2A are stored in the storage space 118a.

Moreover, as shown in FIGS. 37A and B, if the second scope unit 2B is assembled to the fixed unit 3 instead of the first scope unit 2A, the amplification unit 60F is assembled. In this case, by using he storage hollow 117 of the amplification unit 60F and the storage box 118 which is notched on one face, in combination, as shown in FIG. 37B a storage section 131 having a large storage space can be formed.

Here, the following effects are demonstrated in the above structure. That is, in this embodiment, when the normal first scope unit 2A having the shorter insertion section 4a is used, by using the storage box 118 singly in combination with the wall face of the fixed unit 3 and the base unit 4d of the first scope unit 2A, the storage space 118a corresponding to the scope can be formed.

Moreover, if the second scope unit 2B having the longer insertion section 4a is used, by assembling the amplification unit 60F and using the storage hollow 117 of the amplification unit 60F and the storage box 118 which is notched on one face, in combination, as shown in FIG. 37B the storage section 131 having a large storage space can be formed. Therefore, there is the effect of providing a system corresponding to the scope.

(Seventh Embodiment)

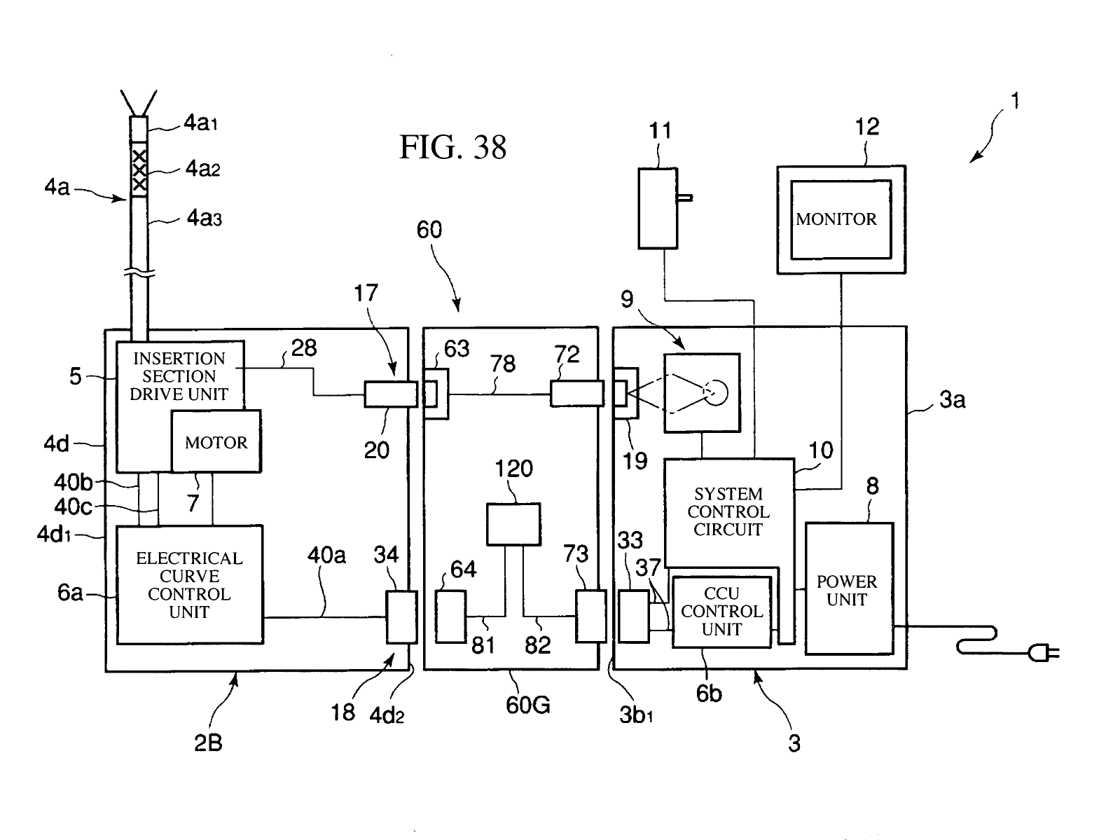

Moreover FIG. 38 shows a seventh embodiment of the present invention. An amplification unit 60G which amplifies the measuring function is provided in this embodiment. A second system control circuit 120 which supports the calculation in parallel with the system control circuit 10 is provided in the amplification unit 60G.

If a binocular adapter which is used for stereoscopic measurement such as the direct sight binocular adapter 100a3 and the side looking binocular adapter 100a4 of FIG. 5 is attached to the tip end face of the head section 4a1 for use, then normally (in the case where the amplification unit 60G is not used) the optical image obtained from both eyes is calculation processed based on parallax by triangulation, by only the system control circuit 10 in the fixed unit 3, so as to obtain the three-dimensional data of the observation image.

In this embodiment, by placing the amplification unit 60G between the scope unit 2 and the fixed unit 3, the calculation to obtain the three-dimensional data is supported not only by the system control circuit 10 but also by the second system control circuit 120 of the amplification unit 60G in parallel with the system control circuit 10. The second system control circuit 120 acts as a proxy for the one part of the calculation processing to obtain the three-dimensional data, being a parallel computer.

Here, the following effects are demonstrated in the above structure. That is, in this embodiment, by placing the amplification unit 60G between the scope unit 2 and the fixed unit 3, there is the effect of performing the calculation faster than that by the conventional system control circuit 10 only, so as to amplify the measuring function.

It should be understood that the above embodiments are not to be considered as limiting the present invention. Additions, omissions, substitutions of the construction, and other modification can be variously performed without departing from the spirit or scope of the present invention. Accordingly, the invention is not to be considered as being limited by the forgoing description, and is only limited by the scope of the appended claims. Furthermore, the respective constructions of the embodiments may be combined or the combination may be modified for use. Particularly, the construction may be such that the system comprises not only one amplification unit but also a combination of two amplification units or more.

1. An endoscope apparatus comprising:
a scope main body which has an insertion section for insertion into a specimen;
a box body section which is used in combination with said scope main body; and
an amplification device which is provided between said scope main body and said box body section, and amplifies said normal function of at least one of said scope main body and said box body section.

2. An endoscope apparatus according to claim 1, wherein
said scope main body is provided with an image sensing device on a tip end thereof, and
said normal function is a function for transmitting and receiving electrical signals to and from said image sensing device.

3. An endoscope apparatus according to claim 2, wherein
said amplification device is a signal amplification device which amplifies said electrical signals.

4. An endoscope apparatus according to claim 3, wherein
said amplification device is an amplification device which amplifies drive signals for driving said image sensing device.

5. An endoscope apparatus according to claim 3, wherein
said signal amplification device is an amplification device which amplifies output signals from said image sensing device.

6. An endoscope apparatus according to claim 3, wherein
said signal amplification device is an amplification device which is provided with a detection device which detects the length of said insertion section, and which sets amplification parameters by means of said detection device, and amplifies said electrical signals based on said set amplification parameters.

7. An endoscope apparatus according to claim 1, wherein
said scope main body is provided with an illumination optical system on a tip end thereof, and
said normal function is an illumination function of said illumination optical system.

8. An endoscope apparatus according to claim 7, wherein
said amplification device is a light quantity amplifying device which amplifies the light quantity of the illumination light of said illumination optical system.

9. An endoscope apparatus according to claim 1, wherein
said normal function is a storage function for storing said scope main body.

10. An endoscope apparatus according to claim 9, wherein
said storage function is a storage section which stores said scope main body.

11. An endoscope apparatus according to claim 10, wherein
said amplification device is a capacity amplification device which amplifies the capacity of one of said storage sections.

12. An endoscope apparatus according to claim 10, wherein
said amplification device is a number of storage sections amplification device which multiply provides said storage sections.

13. An endoscope apparatus according to claim 1, wherein
said normal function is a recording function which records images or sounds detected by said scope main body.

14. An endoscope apparatus according to claim 13, wherein
said box body section comprises a first recording device having said recording function, and
said amplifying device is a second recording device additionally provided in said first recording device.

15. An endoscope apparatus according to claim 1, wherein
said normal function is a curving function for curving a tip end of said scope body and curving it towards a predetermined direction.

16. An endoscope apparatus according to claim 15, wherein
said amplifying device is an electrical curving device which achieves said curving function by electricity.

17. An endoscope apparatus according to claim 15, wherein
said amplifying device is a manual curve drive unit wherein said curving function makes a passive curve due to external force into a main curve.

18. An endoscope apparatus according to claim 1, wherein
said normal function is a measuring function which measures the shape of said specimen three-dimensionally.

19. An endoscope apparatus according to claim 18, wherein
said measuring function is a calculation processing function which performs calculation processing on the optical image detected by said scope main body, to obtain three-dimensional information thereof.

20. An endoscope apparatus according to claim 19, wherein
said amplifying device is a speed enhancing device which enhances the speed of the calculation processing of said calculation processing function.
